(19) United States
(12) Patent Application Publication
(10) Pub. No.: US 2024/0021321 A1
(43) Pub. Date: Jan. 18, 2024

(54) METHODS AND DEVICES FOR ESTIMATING HYDRATION AND HEAT STRESS PHYSIOLOGICAL LEVELS

(71) Applicant: Impact Vitals, Inc., Greenwood Village, CO (US)

(72) Inventors: John Jedziniak, Greenwood Village, CO (US); Abhishek Jaiantilal, Highlands Ranch, CO (US)

(73) Assignee: Impact Vitals, Inc, Greenwood Village, CO (US)

(21) Appl. No.: 18/221,794

(22) Filed: Jul. 13, 2023

Related U.S. Application Data
(60) Provisional application No. 63/388,952, filed on Jul. 13, 2022.
(60) Provisional application No. 63/388,975, filed on Jul. 13, 2022.

(51) Int. Cl. G16H 50/70; A61B 5/0205; A61B 5/00; G16H 50/30

(52) U.S. Cl. CPC G16H 50/70; A61B 5/02055; A61B 5/4875; G16H 50/30; A61B 5/02416

(57) ABSTRACT

Techniques for estimating and/or predicting a hydration index, hydration level change, hydration level, core body temperature index, core body temperature change, and/or core body temperature level noninvasively based on plethysmograph and/or ECG signals.

### RELATED APPLICATION

The present application claims priority to U.S. provisional application No. 63/388,952 filed Jul. 13, 2022 (the “'952 application) and U.S. provisional application No. 63/388,975 filed Jul. 13, 2022 (the “'975 application). The present application incorporates by reference the entire disclosures of the '952 and '975 applications as if each were set forth in full herein.

Dehydration and heat stress are serious health conditions that can cause minor to severe consequences to the human body (“body” for short) whether independently or jointly encountered. Dehydration normally occurs when the body loses more fluids than it takes in, while heat stress occurs when a body's temperature is unable to cool down effectively. The treatments differs for the two conditions, thus it is important to properly differentiate the conditions and the degree of severity.

More particularly, when the body loses more fluids than it takes in, whether through sweating, urination, or other bodily functions dehydration may occur. In hot conditions, excessive sweating can lead to rapid fluid loss which in turn compromises the body's ability to function optimally. Dehydration affects the body's overall well-being, leading to fatigue, dizziness, increased heart rate, and reduced cognitive function. If left unaddressed, severe dehydration can progress to heat exhaustion or heatstroke, life-threatening conditions requiring immediate medical attention.

Heat stress on the other hand arises when the body is unable to dissipate excess heat, causing the body's internal temperature to rise. Prolonged exposure to high temperatures, coupled with high humidity and inadequate ventilation, can overwhelm the body's cooling mechanisms. The signs of heat stress include heavy sweating, muscle cramps, headaches, nausea, and even fainting. If not treated promptly, heat stress can escalate to heatstroke, a medical emergency typically characterized by a core body temperature above 40° C. (104° F.), accompanied by confusion, rapid breathing, and loss of consciousness.

Dehydration and heat stress pose significant dangers to human health, ranging from mild discomfort to life-threatening conditions. The consequences of dehydration extend beyond thirst and discomfort. Mild dehydration can impair cognitive function, diminish physical performance, and exacerbate existing health conditions. In extreme cases, dehydration can lead to hypovolemic shock, kidney failure, and even death.

Heat stress, when not adequately managed, can have severe consequences. Heat stress is the precursor to heatstroke, and if not treated promptly can progress rapidly. Heatstroke is a medical emergency that can cause organ failure, brain damage, and death.

While conditions leading to dehydration and heat stress may occur together (e.g., overexertion of the body in high-heat, high-humid settings), they may also occur independently and the treatment for each condition may be different. For example, dehydration may typically be treated by rehydrating the body through the consumption, or through the administration, of fluids, whereas heat stress may typically be treated by methods that lower the body's temperature. The severity of a dehydration or heat stress condition also dictates the criticality and urgency of administering fluids or reducing the body's temperature using more extreme measures.

Measuring an individual's dehydration levels is typically, most accurately achieved through blood analysis, urine analysis, or carefully monitoring changes in nude body mass. Less accurate methods also exist, such as the use of galvanic skin responses, to estimate a body's fluid (e.g., water) loss through sweat on the skin, or by studying changes in the color of chemicals that are a part of patches placed on the skin and designed to absorb sweat from the skin. Core body temperature levels are typically, most accurately measured through the use of a rectal thermometer or other invasive sensors, though less accurate measurements may also be used, such as using expensive technical solutions that estimate core body temperatures based on changes in a body's skin temperatures.

Accordingly, it is desirable to estimate and predict an individual's hydration level and core body temperature level conveniently, accurately and non-invasively.

### SUMMARY

The inventors describe a number of innovative methods for estimating and predicting the values of hydration and heat stress levels of an individual. For example, one exemplary method for estimating physiological levels may comprise receiving a plurality of electronic signals representing photoplethysmography (PPG) waveforms and/or electrocardiogram (ECG); electronically implicitly or explicitly extracting one or more features of the PPG and/or ECG waveforms from the plurality of electronic signals to generate values indicating one or more physiological characteristics of the PPG and/or ECG waveforms; and electronically correlating the generated values to one or more (i) relative, hydration levels and states, or (ii) relative heat stress levels and states or (iii) a combination of relative, hydration and heat stress levels and states.

Such an exemplary method may further comprise one or more of: (1) electronically extracting the one or more features of the PPG waveforms from the plurality of electronic signals comprises extracting features from one or more categories of features of the PPG and/or ECG waveforms, where the one or more categories of features comprises at least one or more features selected from at least power features, wavelet features, and distribution features of the one or more PPG and/or ECG waveforms; (2) electronically selecting certain segments of the received signals representing certain points that are equidistant apart on each of the PPG and/or ECG waveforms, and further electronically, implicitly extracting one or more features and/or characteristics of the PPG and/or ECG waveforms based on the selected, certain segments to generate the values indicating the one or more physiological effects of the PPG and/or ECG waveforms; (3) electronically excluding corrupted signals within the received electronic signals for an associated time segment during which the corrupted signals were received when one or more extracted features of the received electronic signals and/or an entirety of the received electronic signals is outside an acceptable range as compared to previously observed electronic signals of the same type within a past finite time period stored in an electronic database; (4) electronically adjusting the generated values by skewing, scaling or rotating one or more signals so that the generated values more closely reflect a representation of the received signals representing the PPG and/or ECG waveforms; (5) electronically generating one or more control signals to control the output of the one or more (i) relative, hydration levels and states, or (ii) relative heat stress levels and states or (iii) a combination of relative, hydration and heat stress levels and states or electronically generating one or more alarm indicators indicating one of the one or more (i) relative, hydration levels and states, or (ii) relative heat stress levels and states or (iii) a combination of relative, hydration and heat stress levels and states has exceeded a threshold; (6) electronically receiving personalization data; and further electronically correlating the generated values to one or more (i) absolute, hydration values, or (ii) absolute, skin or core body temperature values, or (iii) a combination of absolute, hydration and skin or core body temperature values.

In addition to innovative methods the inventors provide innovative devices that estimate physiological levels or states. One such device may comprise: one or more electronic processors operable to execute instructions retrieved from one or more electronic memories to: electronically receive a plurality of electronic signals representing PPG and/or ECG waveforms; electronically implicitly or explicitly extract one or more features of the PPG and/or ECG waveforms from the plurality of electronic signals to generate values indicating one or more physiological characteristics of the PPG and/or ECG waveforms; and electronically correlate the generated values to one or more (i) relative, hydration levels and states, or (ii) relative heat stress levels and states or (iii) a combination of relative, hydration and heat stress levels and states.

In such an electronic device the one or more electronic processors may be further operable to execute instructions retrieved from the one or more electronic memories to complete one or more of the following functions: (1) electronically extract the one or more features of the PPG and/or ECG waveforms from the plurality of electronic signals comprises extracting features from one or more categories of features of the PPG and/or ECG waveforms, where the one or more categories of features comprises at least one or more features selected from at least power features, wavelet features, and distribution features of the one or more PPG and/or ECG waveforms; (2) electronically select certain segments of the received signals representing certain points that are equidistant apart on each of the PPG and/or ECG waveforms, and further electronically, implicitly extract one or more features and/or characteristics of the PPG and/or ECG waveforms based on the selected, certain segments to generate the values indicating the one or more physiological effects of the PPG and/or ECG waveforms; (3) electronically exclude corrupted signals within the received electronic signals for an associated time segment during which the corrupted signals were received, when one or more extracted features of the received electronic signals and/or an entirety of the received electronic signals is outside an acceptable range as compared to previously observed electronic signals of the same type within a past finite time period stored in an electronic database; (4) electronically adjust the generated values by skewing, scaling or rotating one or more signals so that the generated values more closely reflect representations of the received signals representing the PPG and/.or ECG waveforms; (5) electronically generate one or more control signals to control the output of the one or more (i) relative, hydration levels and states, or (ii) relative heat stress levels and states or (iii) a combination of relative, hydration and heat stress levels and states or electronically generate one or more alarm indicators indicating one of the one or more (i) relative, hydration levels and states, or (ii) relative heat stress levels and states or (iii) a combination of relative, hydration and heat stress levels and states has exceeded a threshold; (6) electronically receive personalization data; and further electronically correlate the generated values to one or more (i) absolute, hydration values, or (ii) absolute, skin or core body temperature values or (iii) combination of absolute, hydration values, and skin or core body temperature values.

### BRIEF DESCRIPTION OF THE DRAWINGS

FIG. 1 depicts a simplified block diagram of an innovative, referential process or method (hereafter the two words may be used interchangeably) for estimating hydration, heat stress and combined hydration and heat stress reference values based on the transformation of historical, physiological data according to embodiments of the disclosure.

FIG. 2 depicts a simplified block diagram of an innovative method for estimating current, real-time values of hydration and heat-stress levels of a current individual and predicting future estimates of hydration and heat-stress levels for such an individual according to embodiments of the disclosure.

FIG. 3 depicts an exemplary normalization process for a physiological waveform used to explain features of the present disclosure.

FIG. 4 depicts an exemplary, representative PPG waveform used to explain features of the present disclosure.

FIG. 5A depicts an exemplary PPG waveform, while FIGS. 5B and 5C depict curves representing power spectral densities (PSD) for a segment of the PPG waveform (FIG. 5B), and for the entire PPG waveform (FIG. 5C). FIG. 5D depicts the PSDs from FIGS. 5B and 5C overlaid or overlapped on one another.

FIG. 6A depicts another exemplary PPG waveform while FIGS. 6B to 6F depict decomposed representations of the waveform in FIG. 6A.

FIGS. 7 to 9 depict bar graphs of innovative hydration index, heat stress index and a combined hydration and heat stress index, respectively, for different physiological states according to embodiments of the disclosure.

FIG. 10 depicts an exemplary three dimensional representation (e.g., a planar 3D contour) that may be used to predict a percentage of fluid weight loss based on an exemplary predictive, interpretative process.

FIG. 11 depicts an exemplary three dimensional representation (e.g., a planar 3D contour) that may be used to predict changes in a core body temperature based on the exemplary predictive, interpretative process.

FIG. 12 depicts a Bland-Altman plot that compares predicted body weight loss percentages versus actual body weight loss percentages due to water loss based on the interpretative process.

FIG. 13 depicts a Bland-Altman plot that compares predicted core body temperatures versus actual core body temperatures based on the interpretative process.

### DETAILED DESCRIPTION

Exemplary embodiments of methods and devices for estimating and predicting values that correspond with hydration and heat stress levels of an individual are described herein and are shown by way of example in the drawings. Throughout the following description and drawings, like reference numbers/characters may refer to like elements.

It should be understood that although specific embodiments are discussed herein, the scope of the disclosure is not limited to such embodiments. On the contrary, it should be understood that the embodiments discussed herein are for illustrative purposes, and that modified and alternative embodiments that otherwise fall within the scope of the disclosure herein are contemplated.

As used herein, the words “comprising”, and any form thereof such as “comprise” and “comprises”; “having”, and any form thereof such as “have” and “has”; “including”, and any form thereof such as “includes” and “include” are inclusive or open ended and do not exclude additional, unrecited elements, functions or process steps.

As used herein, the term “a” or “an” may mean “one”, but may also mean “one or more”, “at least one”, and “one or more than one” depending on the usage and context.

It should also be understood that one or more exemplary embodiments may be described as a process or method. Although a process/method may be described or depicted as sequential, it should be understood that such a process/method may be performed in parallel, concurrently or simultaneously. In addition, the order of each step within a process/method may be re-arranged. A process/method may be terminated when completed and may also include additional steps not included in a description or depiction of the process/method.

As used herein, the term “and/or” includes any and all combinations or permutations of one or more of the associated listed items.

It should be understood that when one part or step in an innovative device or method is described or depicted as being “connected” to another part or steps, other parts or steps used to facilitate such a connection may not be described or depicted because such parts or steps are well known to those skilled in the art.

Yet further, when one part or step of a device or method is described or depicted as being connected to another part or step in a figure it should be understood that, practically speaking, such a connection may comprise (and many times will comprise) more than one physical connection or processing step.

It should be noted that the devices, as well as any components, or elements thereof, illustrated in the figures are not necessarily drawn to scale, and need not be representative of an actual shape or size and need not be representative of any actual device. Rather, the devices, components and elements are drawn so as to help explain the features, functions and processes of various exemplary embodiments of the described disclosure.

Relatedly, to the extent that any of the figures or text included herein depicts or describes operating parameters or waveforms it should be understood that such information is merely exemplary and non-limiting, and is provided to enable one skilled in the art to practice exemplary embodiments of the disclosure.

Where used herein, the letter “n” may denote the last step or component of one or more steps or components (e.g., sensors 3a to 3n).

As used herein the term “signal data” means physiological signals of a human heart detected and/or collected by physical sensors of various kinds (electronic, electromechanical, etc.) from the bodies of individual persons related to blood flow. Some non-limiting examples of signal data are heart pulse rates, blood pressures, blood oxygenation levels, electrocardiogram (ECG) data, photoplethysmography (PPG) data (e.g., data derived from samples of measured, heart rate waveforms) and the like.

As used herein the term “non-signal data” means data that is not signal data.

As used herein the term “ambient temperature” means the measured temperature or the current temperature of the environment surrounding an individual while physiological data was being measured and collected from the individual. As used herein, the temperature of an individual's body (e.g., rectal temperature, skin temperature) is not an ambient temperature (hereafter may be referred to as “body temperature”).

As used herein the term “physiological state” means a condition that can be inferred from physiological signals or related data, but which is, per se, directly measurable. Examples of physiological states include dehydration, heat stress, and temperature shock. For purposes of prevention, diagnosis, and treatments, it is useful to sense and collect (i.e., monitor) data that indicates the existence of potentially harmful physiological states, and to monitor data regarding the severity of such states.

As used herein the term “heat stress” or “heat” means one or more physiological states or levels, including, but not limited to physiological states or levels comprising elevated body temperature(s), core body temperature(s), skin body temperature(s) and temperatures that may occur at any body temperature, including normal or elevated temperatures.

As used herein, the term “hydration” means one or more physiological states or levels, including, but not limited to, dehydration, a physiological state of decreased water fluid in an individual's body, and may include (depending on the context) a euhydrated (fully hydrated) state (i.e., a 0% dehydrated).

As used herein the terms electronic processors “operable to” (e.g., one or more electronic processors 2a to 2n, or 4a to 4n) or similar phrases means that the one or more electronic processors function to execute electronic instructions retrieved from their electronic memories (not shown in Figures) to complete one or more functions of an innovative device(s) or one or more steps of an innovative method(s).

As used herein, the terms “embodiment” or “exemplary” refer to a non-limiting example of the present disclosure.

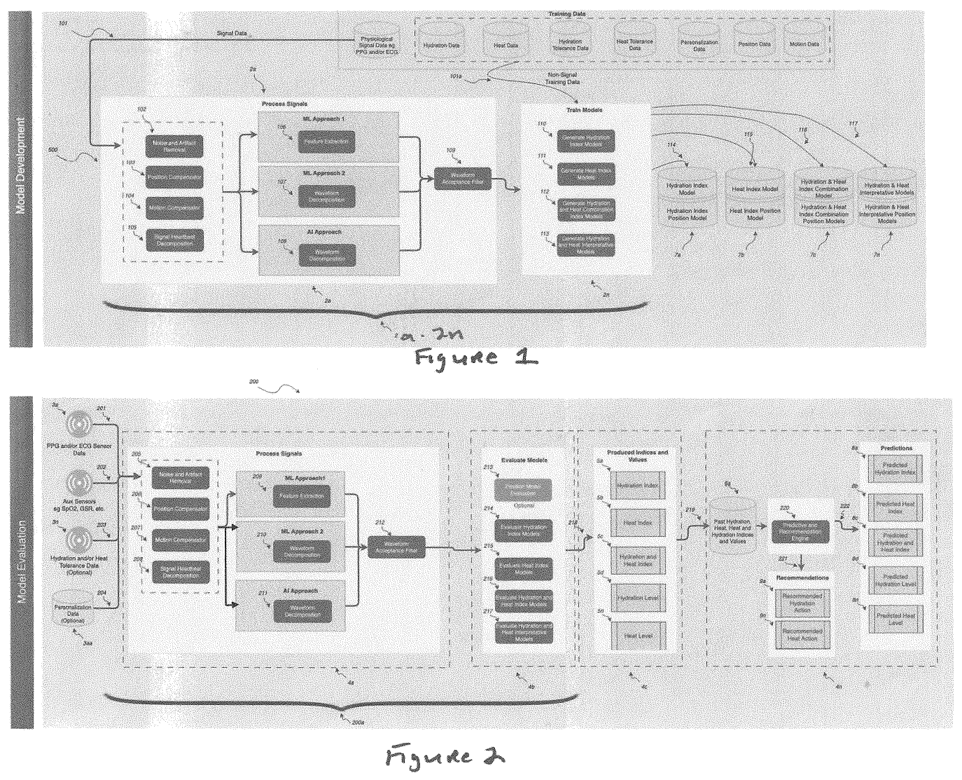

Referring now to FIG. 1 there is depicted a simplified block diagram of an innovative, referential method 100 used to develop a referential, physiological process for estimating values of hydration and/or heat stress levels of an individual according to an embodiment of the disclosure.

For the reader's benefit, one aspect of the present disclosure is to set forth the details of innovative methods that may be used to indicate the estimated, current and future (i.e., predicted) values of hydration and heat stress levels of individuals. The inventors believe the innovative methods discussed herein are more robust than existing methods.

Referring now to FIG. 1, a first step 101 in a referential process or method 100 may comprise receiving a plurality of sampled electronic signals representing historical, signal data at one or more electronic processors 2a to 2n. Such data may be sent from one or more electronic storage devices 1a to 1n (e.g., database(s), electronic servers). In addition, in step 101a the one or more electronic processors 2a to 2n may receive historical, non-signal data from one or more electronic storage devices 1b to 1n (e.g., database(s), electronic servers)(where ‘n” indicates a last storage device).

In embodiments, the signal data may comprise ECG data, PPG data and/or other data derived from blood flow while the non-signal data may comprise historical data, including, but not limited to, hydration data, heat stress data, hydration tolerance data, heat tolerance data, position data and motion data that have been previously correlated to historical waveforms or waveform components of a human heart (e.g., PPG or electrocardiogram (ECG) waveform or waveform components have been previously correlated to hydration levels, heat stress levels, etc.). The non-signal data may further comprise personalization data as will be described further herein.

Collectively, the signal and non-signal data may be referred herein to as “referential historical, physiological data” with respect to process or method 100 (or simply “referential data”).

It should be understood that the number of storage devices 1a to 1n shown in FIG. 1 is merely exemplary. Accordingly, referential data may be stored in a fewer, or greater, number of storage devices as shown in FIG. 1.

In embodiments, the signal data may be derived from sensors that detect and measure signals representing PPG or ECG waveforms of individual volunteers, to name just two examples of such signals, (e.g., individuals involved in tests, experiments or those individuals whose levels were measured in the past) that were subject to varying levels of hydration (with and without varying levels of mineral water supplementation), varying levels of heat stress (including varying levels of temperature and humidity), varied motion (e.g., walking, running, various physical exercises and/or activities) and/or varied positions (e.g., lying down (face up, sitting, standing) while the non-signal data may be derived from responses to one or more questionnaires, electronical health records, including historical records of data representing an individual's tolerance to changes in hydration levels (“hydration tolerance data”), and tolerance to changes in temperature levels (“heat tolerance data”).

In embodiments, some exemplary examples of personalized data include information provided by an individual involved in an experiment or test regarding tolerance to exercise activities (e.g., are you out of breath after running up a flight of stairs?), a physical condition, age, sex, height, weight, body mass index, body fat percentage, VO2max, exercise frequency, exercise intensity, garments (e.g., does the individual usually wear a hat? i.e., because wearing a hat may retain more body heat), hair length (individuals with long hair may retain more body heat), geographical location, past or recent exposure to ambient temperatures, medications, disease, and/or illnesses.

In slightly more detail, additional examples of signal data may be data that is derived by determining water loss(es) and core body temperatures of an individual at various ambient temperatures and motions, in addition to capturing the resulting heart waveforms (e.g., PPG waveforms, ECG waveforms) associated with each ambient temperature and motion (i.e., intensity of exercise) of the individual and extracting components of, or processing all of, the resulting heart waveforms and storing the extracted components or waveforms (i.e., electronic signals) within the one or more electronic storage devices 1a to 1n.

Similarly, other types of signal data may be derived by determining water loss(es) and core body temperatures that represent an individual under various ambient temperatures and exercising at certain intensities, speeds, or surrounding times, or is positioned at a certain angle from a horizontal or vertical referential axis, for example, by capturing the resulting heart waveforms (e.g., PPG waveforms, ECG waveforms) associated with each ambient temperature/motion/position (e.g., sitting, standing, supine), and extracting components of, or processing all of, the resulting heart waveforms and storing the extracted components or waveforms within the one or more electronic storage devices 1a to 1n.

Non-signal data may also include stored physiological data representing electronic measurements previously made by one or more sensors that may have been used to monitor a plurality of individuals (one at a time) under various temperatures, humidity, motions and positions. Such data may be stored in the one or more electronic storage devices 1a to 1n. For example, such data may comprise physiological data previously collected from individuals involved in previous experimental trials or studies that subjected the individuals to various levels of hydration, heat stress, motion and/or position and correlated such levels to characteristics of a heart waveform (e.g., PPG, ECG waveform).

Continuing, upon receiving signal data from electronic storage devices 1a to 1n in step 101 the one or more electronic processors 2a to 2n involved in completing steps of method 100 may be operable to retrieve instructions stored in their electronic memory (not shown) in order to transform (i.e., correlate) the received signal data to estimated value(s) of hydration and heat stress levels using additional steps 102 to 113 described herein that may be included in method 100.

Continuing, in an embodiment the one or more electronic processors 2a to 2n may be operable to optionally transform the received signal data into an improved representation of the original heart waveform data from electronic storage devices 1a to 1n by electronically removing unwanted or undesirable electronic noise or artifacts (representing erroneous signals or data) from the data in step 102 using, for example, one or more analog or digital electronic filters or filtering processes, thereby increasing the accuracy of the received data, and eventually, the correlated estimates of a hydration level and heat stress levels, among other levels.

The received signal data may (as an option) include motion and position data components indicating the relative or particular motion and position of an individual while at a particular hydration and heat stress level. Such position data may have been manually captured or automatically detected by positional sensors, for example, while the motion data may have originated from multi-dimensional accelerometers, gyroscopes, and multi-body-location physiological sensors, for example.

Accordingly, the one or more electronic processors 2a to 2n may be operable to optionally, electronically transform (i.e., adjust) the received signal data by adjusting the data based on the position of the individuals in step 103.

Further, the one or more electronic processors 2a to 2n may be operable to further transform the received data by optionally, electronically adjusting the data based on motion of the individual in step 104.

In more detail, unwanted, electrical noise in the received signal data may be caused by different motions or positions (e.g., walking versus running versus, repetitive motions and/or standing versus sitting versus supine) made by the individuals connected to sensors that were involved in experiments or tests. Accordingly, in optional step 103 and/or 104, to compensate for such a set of diverse noise sources the electronic processors 2a to 2n may be operable to optionally, electronically cancel such unwanted noise by electronically adding or subtracting stored compensation values, for example. Alternatively, instead of adjusting the data using processors 2a to 2n, the data may be initially adjusted before being stored in electronic storage devices 1a to 1n by using accelerometers, multiple PPG sensors or other sensors that are configured to remove the unwanted noise derived from different motions and positions used by individuals to generate the signal data. Still further, rather than adjust the signal data before signal processing steps 106 to 108, the signals may be adjusted after during steps 110 to 113. For example, the one or more processors 2a to 2n may execute stored instructions retrieved from their memories (not shown) to complete a signal mapping process that maps (i.e., assigns) signal data representing one or more time-aligned segments of a waveform (e.g., PPG, ECG waveform) to one or more time-aligned segments of another waveform as explained further in co-pending U.S. patent application Ser. No. ______, the contents of which are incorporated by reference herein as if set forth in full herein. Yet further, other alternative adjustment or filtering processes may be used, such as (a) adjusting the values representing a PPG waveform based on AI or machine learning (ML) processes that have been “trained” (i.e., developed) using PPG adjustment values for the different positions and types of PPG signals, or (b) completing additional ML or AI processes to determine index or value adjusters following steps 110 to 113.

Continuing, upon completing one or more of optional steps 102 to 104, the signal data may then be further transformed by decomposing the data into one or more different, constituent data sets representing constituent heart signals (i.e., waveforms) or components in step 105.

For example, in one embodiment the one or more electronic processors 2a to 2n may be operable to identify and process data representing PPG waveform signals from the data in step 105. In yet another embodiment electronic processors 2a to 2n may be operable to identify and process data representing ECG waveform signals in step 105 to name just two types of heart waveform signals that may identified and processed.

For ease of explanation, for purposes of the following discussion we will select PPG waveform signals as the signals of interest though, again, this is merely exemplary and non-limiting (i.e., the signals can be different than PPG signals, e.g., ECG signals).

Continuing, during step 105 the one or more electronic processors 2a to 2n may be operable to identify constituent PPG waveform components within the signal data. Such data (or data segments) may, for example, represent a period of time corresponding to the heartbeat of an individual (e.g., data indicative of the start and end periods of the heartbeat) and may include past heartbeat detection information retrieved from an electronic memory or storage component for predicting the likely time occurrence of a subsequent heartbeat (memory/storage component not shown for simplicity sake).

In addition, in an embodiment, if the signal data is based on “poor” signals, noisy signals, interference, or another reason (i.e., the data may not satisfy certain, detectable threshold levels)(hereafter collectively referred to as “corrupted data’), such corrupted data may prevent the one or more electronic processors 2a to 2n from clearly identifying a representative heart waveform or waveform component (e.g., a PPG or ECG waveform). Accordingly, in such an instance the processors 2a to 2n may be operable to exclude such corrupted data from further processing for an associated time segment. Accordingly, this function of the processors 2a to 2n effectively acts as an electronic filter of corrupted data received over heartbeat time segments.

Continuing, upon completion of the signal decomposition of the data in step 105, the decomposed data or data segments may then be further transformed into one or more different data values by the one or more electronic processors 2a to 2n, in steps 106, 107, and/or 108, depending on the innovative, signal processing ML or AI process selected. In embodiments, the one or more processors 2a to 2n may retrieve electronic signals stored as instructions in their electronic memories for executing one or more of the processes represented by steps 106, 107 and.or 108 in FIG. 1, said another way, a device (e.g., mobile device, PC, server) may be programmed to include instructions to execute the process in step 106, 107 or 108 or some combination of steps 106, 107 and 108.

In more detail, in step 106 a first ML process (referred to herein as “first ML Process” or “ML Approach 1” in FIGS. 1 and 2) may generate values that correspond to certain heart waveform signal features of the signal data (also known as “feature extraction”) through the execution of electronic instructions by the one or more electronic processors 2a to 2n (stored in their electronic memory, not shown). The generated values of the so extracted features may then be correlated to physiological states as explained further herein. The generated values may be electronically stored and utilized for later analysis.

Alternatively, in step 107 a second ML process (referred to herein as “second ML Process” or “ML Approach 2” in FIGS. 1 and 2) may be completed by the one or more electronic processors 2a to 2n through the execution of electronic instructions stored in their electronic memory. This second ML process may include steps that further transform the data from step 105 to generate interpolated values derived from equidistant points of a heart waveform. In an embodiment, such values may then be electronically stored and utilized for later evaluation.

For the reader's benefit, the second ML process may be viewed as related to the the first ML process except that instead of generating values based on a large number of data points representing a heart waveform signal, features are implicitly extracted from which values are generated based on selecting certain points on a heart waveform signal that are equidistant apart. It is believed that such a selection reduces the computational complexity of the second ML process as compared to the first ML process.

In yet another alternative process, in step 108 the one or more electronic processors 2a to 2n may execute electronic instructions stored in their electronic memory to complete a series of artificial intelligence or “AI” process steps (“AI process” or “AI Approach” in FIGS. 1 and 2). In an embodiment, when an AI process is incorporated into the referential process or method 100 such a process or method may include convolutional neural network processing or recurrent neural network processing or other neural network based or equivalent processes (e.g., algorithms), among other things, to further transform the received data into detectable patterns of data that can be further correlated to physiological states.

As discussed further herein, the AI process may implicitly extract features of a heart waveform signal and/or waveform components that may be correlated to a heat or hydration index value.

For the reader's benefit, and to describe possible, exemplary “use cases” for the different process in steps 106 to 108, the exemplary first ML process may be advantageously used when it is desirable to know, and trace, the type of features that have been extracted form a heart waveform signal (i.e., traceability), such as may be necessitated in medical related settings, while the exemplary second ML process may be used when traceability is not a concern. In embodiments, completing the second ML process may require less computational energy (e.g., power, and, therefore, may provide an electronic device with a longer battery life) for processors 2a to 2n or any associated device as compared to the first ML process, and thus would be well suited for lower processing platforms such as wearable devices and phones. Further, the exemplary AI process may require the most computational energy to complete its processes and necessitate a substantial amount of heart waveform signal data, for example, as may be available in a patient Electronic Health Record (EHR) system. Because the exemplary AI process in step 108 may comprise a large neural network process, the AI process may be able to detect or determine more subtle differences as it correlates signa data. Accordingly, the exemplary AI process may be more suitable to electronic server or “cloud”-based environments where large data sets and computational processing power is typically available.

It should be understood that each of the first ML process (step 106 in FIG. 1, and step 209 in FIG. 2), second ML process (step 107 in FIG. 1, and step 209 in FIG. 2) and AI process (step 108 in FIG. 1, and step 210 in FIG. 2) comprise electronic signal processing processes.

Continuing, the first ML process may identify and generate values that correspond to certain heart waveform signal features, for example PPG waveform signal features. In embodiments (and as explained further below in the EXPERIMENTS section) during step 106 (and step 209 described below) the one or more electronic processors 2a to 2n may identify over 3,000 features of a single PPG waveform (“Raw PPG waveform data”). Upon identifying and computing the values of the Raw PPG waveform data the first ML process may be further operable to transform such data into “Normalized PPG waveform data”. By transforming the Raw PPG waveform data into Normalized PPG waveform data inconsistencies inherent in the data may be minimized.

For example, suppose the values representing signal data (e.g., Raw PPG waveform data) stored in storage devices 1a to 1n was originally measured using many diverse medical devices/systems, where each device/system used a different unit of measurement to indicate the values of the amplitude of a PPG waveform. In such an occurrence it may be difficult and computationally undesirable to effectively analyze such diverse Raw PPG waveform data and its associated values.

Accordingly, to reduce errors in analyzing such Raw PPG waveform data the values representing the diverse Raw PPG waveform data may be transformed into normalized values (i.e., Normalized PPG waveform data).

For example, suppose one device/system has measured the amplitude of a PPG waveform over a fixed time period and indicates the amplitude has a value between and 38,000 units while another device/system measures the same PPG waveform over the same time period and indicates the amplitude has a value between 10,000 and 18,000 units. Thus, the identical PPG waveform has been measured using two diverse and different units of measurement. In an embodiment, the first ML process would transform or convert each of the values (V) measured by the two devices/systems into a normalized unit of measurement between 0 and 1, for example, by determining the minimum and maximum amplitude values (Vₘᵢₙ and Vₘₐₓ, respectively) and computing the normalized value with the formula (V−Vₘᵢₙ)/Vₘₐₓ for the value V.

For example, suppose an actual measured value of a PPG waveform is 32,000 and during a given time period the minimum value is 30,000 and the maximum value is 38,000, then the normalized value would be 0.25 (i.e., 32,000−30,000)/(38,000−30,000)=0.25.

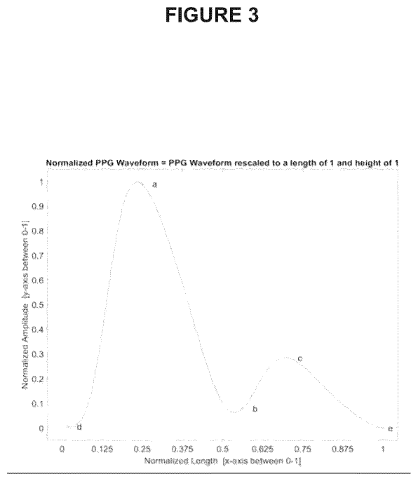

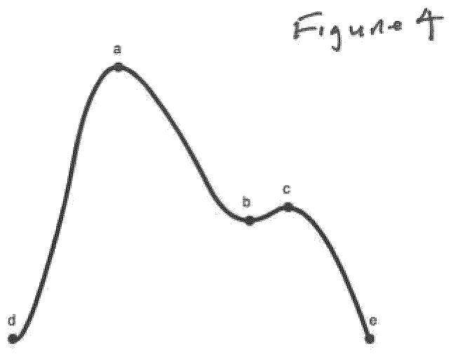

Normalization may also occur over the horizontal dimension, or time period, of the waveform using a similar approach. An example of a waveform that is normalized for the X and Y axis is shown in FIG. 3.

The inventors believe that the transformation or conversion of measured PPG waveforms to a normalized unit of measurement generates more consistent predictions (i.e., predictive values) especially when PPG waveforms may be sampled at different sampling rates (e.g., 25 Hertz (Hz) versus 75 Hz) and are of different lengths due to varying heart rates.

To illustrate the extraction of features from a PPG waveform reference will be made to FIGS. 3 and 4.

The first category of features that may be extracted from the signal data representing a PPG waveform by the first ML process during step 106 (and step 209 described below) may be referred to as “location features”. In embodiments, location features may relate to the time or sequence number, or location of a specific point, points or characteristics of a PPG waveform. For example, the location of the points of the PPG waveform that are associated with the systolic peak, dicrotic notch, and diastolic peak identified as points “a”, “b”, “c” respectively in FIG. 4 are location features. Other “location” features may include additive values, subtractive values, ratios, and other types of values that are determined based on the location of two or more points on a PPG signal, for example, where such values may indicate a characteristic of a PPG waveform.

In embodiments, the values associated with location features of a heart waveform (e.g., PPG waveform, ECG waveform) may be converted from Raw PPG (or, e.g. Raw ECG) waveform values into Normalized PPG waveform values in step 106. Some examples of location features whose values may be identified and generated are as follows:
a. the values at locations “a”, “b”, “c” (i.e., using Raw PPG waveform values);
b. the values at normalized locations “a”, “b”, “c”: (i.e., using Normalized PPG waveform values);
c. combined values at locations given by: f(a,b), f(b,c), f(a,c), where, in more detail, f(a,b) equals the value at point “a” minus the value at point “b” (i.e., using Raw PPG waveform value or Normalized PPG waveform values;

The second category of features that may be extracted (i.e., identified, values generated) from signal data representing a PPG waveform by the first ML process during step 106 may be referred to as “amplitude” features.

In an embodiment, the values of the amplitude of a PPG waveform at specific locations (e.g., locations “a” through “e” in FIGS. 3 and 4) may be identified. For example, the value of the amplitude at the systolic peak, dicrotic notch, and diastolic peak (points a, b, c respectively in FIGS. 3 and 4) may be identified, or, alternatively, the amplitude at a specific point may be represented as a value equal to a ratio of the PPG waveform's total amplitude. Furthermore, additive amplitude values, subtractive amplitude values, amplitude ratios, and other types of amplitude values that are based on two or more points on a PPG waveform may be used (using either Raw PPG waveform values and/or Normalized PPG waveform values). Some examples of amplitude features are as follows:
a. amplitude vales at “a”, “b”, and “c” based on Raw PPG waveform values;
b. amplitudes at “a”, “b”, “c”: For each point, a minimum amplitude of the waveform is determined based on Raw PPG waveform values;
c. normalized amplitude of “a”, “b”, “c” based on Normalized PPG waveform values.
d. amplitudes determined by the combination of points such as f(a,b), f(b,c), f(a,c), where, for example, f(a,b) equals the amplitude value at point “a” minus the amplitude value at point “b” using both Raw and/or Normalized PPG waveform values.

It should be understood that as necessary, the values associated with amplitude features of a heart waveform (e.g., PPG waveform, ECG waveform) may be converted from Raw PPG waveform amplitude values into Normalized PPG waveform amplitude values during step 106 (and step 209).

The third category of features that may be extracted from signal data representing a PPG waveform by the first ML process during step 106 (and step 209) may be referred to as “slope” features.

In an embodiment, the computed slope of a line connecting pairwise points of a PPG waveform may be determined, where the slope may be defined as the ratio of the change in the value of the amplitude between two points of a PPG waveform and the corresponding change in the value of the location for the same two points. For example, the slope between point “a” and “b” in FIGS. 3 and 4 may be given by the computed difference in the amplitude value at point “b” minus the amplitude value at point “a” divided by the computed difference of the value of the location at point b minus value of the location at point a.

Furthermore, the additive slope values, subtractive slope values, slope ratios, and other types of slope values based on two or more such points may be used (using either Raw PPG waveform values and/or Normalized PPG waveform values). Some examples of slope features are as follows.
a. slope: f(a,b), f(b,c), f(a,c) (e.g., f(a,b)=the difference in amplitude between points a and b divided by the difference in location between points a and b.
b. combinations: g(a,b,c) (e.g., g(a,b,c) equal to the value of the slope between points (a,b) on a PPG waveform added to the value of the slope between points (b,c) of the same PPG waveform and the value of he slope between points (a,c) of the same PPG waveform;

The fourth category of features that can be extracted by the first ML process from signal data representing a PPG waveform in step 106 (and step 209) can be referred to as “area” features: For example, the area defined by the following:
a. Area Set 1: The values of the areas (based on Raw PPG waveform values and/or Normalized PPG waveform values) under the curve associated with a PPG waveform, between identified points “a”, “b”, “c”, “d”, and “e” in FIGS. 3 and 4. Also, the ratio of the values of each of the areas for each of the pairwise combination of these points.
b. Area Set 2: The values of the areas (based on Raw PPG waveform values and/or Normalized PPG waveform values) under the curve associated with a PPG waveform, from the start of a PPG waveform to various points of the first and second derivative of the PPG waveform, including derivatives at identified points “a”, “b”, “c”, “d”, and “e” in FIGS. 3 and 4. Also, the ratio of the values of each of the areas for each of the pairwise combination of these points.
e. Combination of Area Sets: For each of the Area Sets discussed above, the combination of two or more such area values, including identified points “a”, “b”, “c” in FIG. 4 (e.g., f(a,b,c), where f(a,b) could be equal to the area between point “a” and point “b” divided by f(b,c) that could be the area between point “b” and point “c”;

The fifth category of features that may be extracted from signal data representing a PPG waveform by the first ML process during step 106 (and step 209) may be referred to as “derivative” features. In embodiments, values representing single and multiple derivative curves of features of a PPG waveform may be computed, along with related features, based on utilizing Raw PG waveform data and/or Normalized PPG waveform data. The values that may be completed include, but are not limited to, the following:
a. computing a value of a PPG feature described previously above representing a first derivative maximum value minus a first derivative minimum value;
b. computing a value of a PPG feature described previously above representing a second derivative maximum value minus a second derivative minimum value;
c. computing a value of a PPG feature described previously above representing the first derivative and second derivative values of a PPG waveform, for example, computing an amplitude value for the first derivative and second derivatives as described above;

The sixth category of features that may be extracted from signal data representing a PPG waveform by the first ML process during step 106 (and step 209) may be referred to as “power” features. In embodiments, one type of power feature that may be extracted is a value representing a power spectral density (PSD) for a portion of a PPG waveform between all combination of pairs of points within the portion. Further, a second type of power feature representing a value of a PSD for a derivative curve described previously above may be computed for a portion of a PPG waveform between all combination of pairs of points.

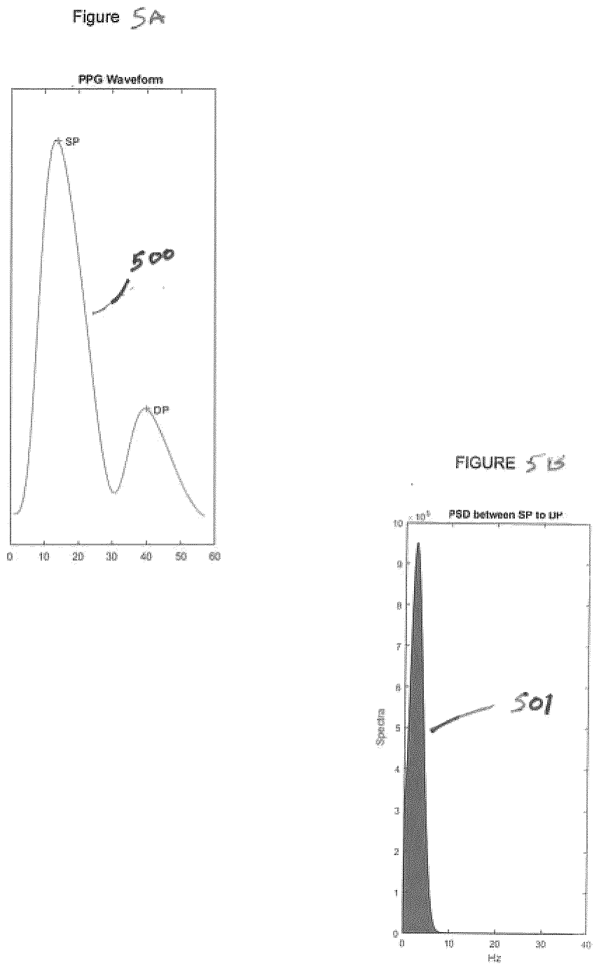

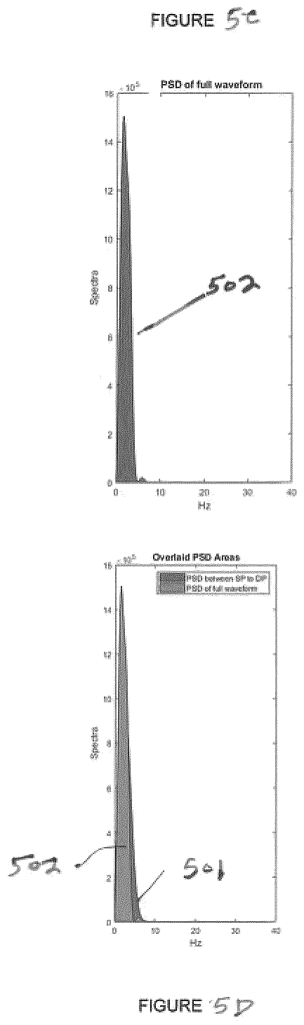

For the reader's benefit we now refer to FIGS. 5A to 5D. These figures depict an exemplary computation of a PSD, it being understood that PSD is only one type of power feature that may be computed by method 100 during step 106 (and method 200 during step 209 described below). FIG. 5A depicts an exemplary PPG waveform 500, FIG. 5B depicts a curve 501 representing a PSD (i.e., represented by the area under the curve 501) that was computed for a segment of the waveform 500 between points SP and DP (i.e., between a pairwise set of points) while FIG. 5C depicts a curve 502 representing the PSD for the entire waveform 500. Finally, FIG. 5D depicts both curves 501 and 502, where curve 501 overlaps curve 502 (or vice-versa). In embodiments, during method step 106 (and 209) an associated device or devices may generate (i.e., compute) the areas under curves 501 and 502 (i.e., PSDs), as well as generating the difference (in area, i.e., in PSDs either overall area or between area bounded by frequency in Hz) between each curve 501,502 and a ratio of the areas of curves 501, 502.

The seventh category of features that may be extracted from signal data representing a PPG waveform by the first ML process during step 106 (and step 209) may be referred to as “distribution” features. In an embodiment, many values representing different distribution features may be computed, including but not limited to values representing a variance, skewness and kurtosis characteristics of a PPG waveform between all pairwise combinations of points on a PPG waveform (i.e., using values derived from location features described above), for all amplitude values described above, and for all derivative values described above.

In more detail, to further describe the innovative distribution features that may be extracted from a PPG waveform by the first MLprocess during step 106 (and step 209) we will use the term “moment” to describe a computational function. Accordingly, a “first moment” may represent a function's expected value, a “second moment” may represent a variance in the expected value, a “third” moment may represent a skewness value, while a “fourth moment” may represent a kurtosis value. Assume that X is a function that is represented by a sequence of values then,
a variance value is typically computed based on the following relationships: E[(X−μ)²], where X is a sequence, and μ is it's mean;
a skewness value is typically computed based on the following relationships:

$$E [ {( \frac{X - μ}{σ} )}^{3} ] ,$$

where X is the variable, μ is it's mean and σ is the standard deviation; and a kurtosis value is typically computed based on the following relationships:

$$E [ {( \frac{X - μ}{σ} )}^{4} ] ,$$

where X is sequence, μ is the mean, and σ is the standard deviation.

The values representing the above moments and additional moments (i.e., so-called “higher” moments) may be computed by one or more electronic processors 2a to 2n in step 106 (or by processors 4a to 4n in step 209) for a PPG waveform or component of a PPG waveform by treating the waveform as X, a sequence of values.

The eighth category of features that may be extracted from signal data representing a PPG waveform by the first ML process during step 106 (and step 209) may be referred to as “wavelet features”. Those skilled in the art will understand that the term “wavelet” refers to a mathematical function that may be used in a process that analyzes signals and data as a part of a signal processing process. Unlike traditional Fourier analysis, which decomposes a signal into sine and cosine waves of different frequencies, wavelet analysis breaks down a signal into wavelets of different scales and positions. The wavelets are localized in both time and frequency domains, allowing for a more precise analysis of transient and localized features in a signal.

In embodiments, statistical computations may be generated for various wavelet representations based on Raw PPG waveform data and Normalized PPG waveform data, including, but not limited to, statistical “means”, and “quantiles” at multiple levels of discrete and continuous wavelet decompositions (i.e., discrete decomposition is typically completed in j levels, where in each level you cut the frequency analysis by half while continuous decomposition allows for finer “grain” control for frequency analysis due to the completion of more computations beyond arbitrary levels).

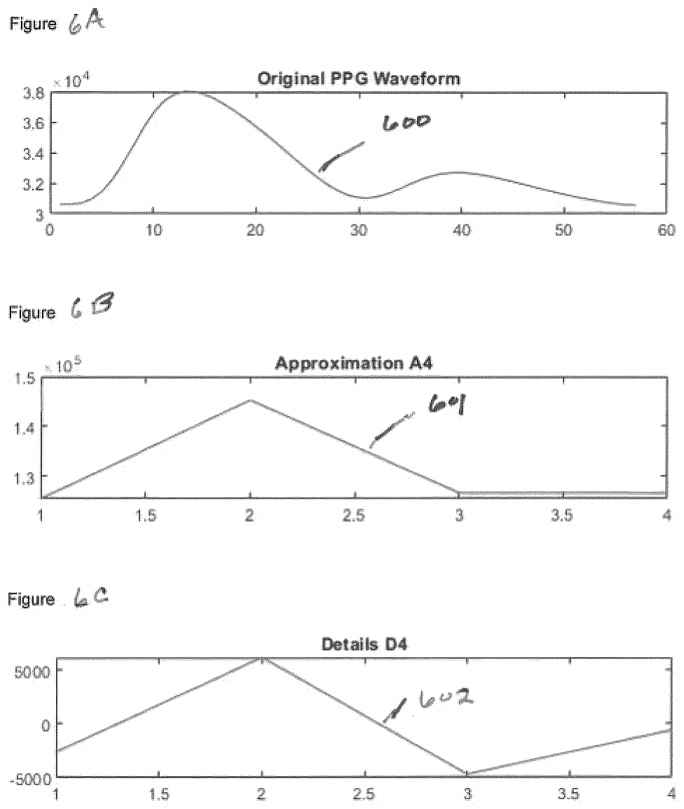

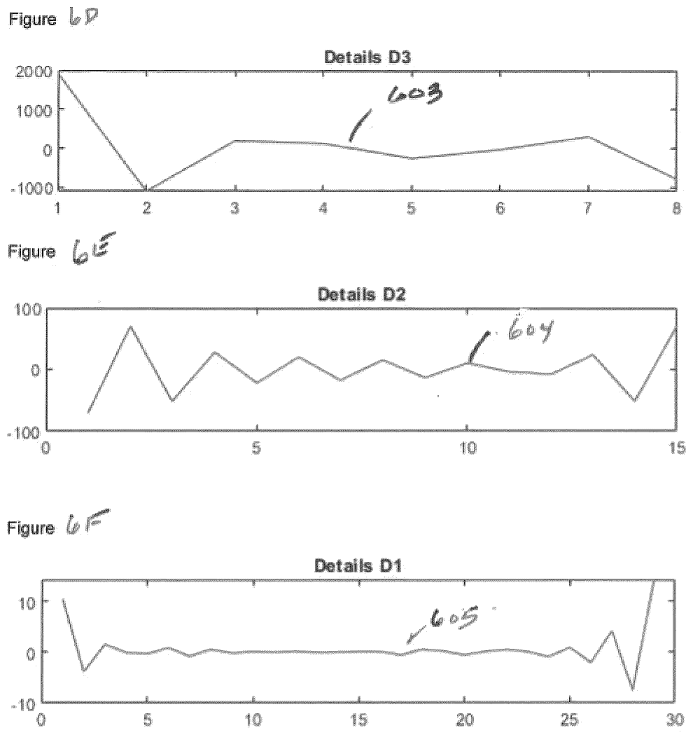

For example, FIG. 6A depicts another exemplary PPG waveform 600 while FIGS. 6B to 6F depict decomposed wavelet representations 601 to 605 of waveform 600 with approximate and coefficients shown in FIG. 6B to FIG. 6E utilizing a Discrete Meyer or “dmey” wavelet over 4 levels. In embodiments, during step 106 (and step 209) statistical means, quantiles, and other statistical values may be computed for each decomposed representation 601 to 605 of waveform 600.

Of course, FIGS. 6A to 6F depict only representations of a single PPG waveform and its related values while method 106 (and 209) may generate (i.e., compute) statistical computations (values) for a plurality of PPG waveforms.

The ninth category of features that may be extracted from signal data representing a PPG waveform by the first ML process during step 106 (and step 209) may be referred to as “width” features. In an embodiment, the width (i.e, difference along the X-axis) of a PPG waveform may be computed between multiple points of the waveform. For example, a width may be computed (using Raw PPG values or Normalized PPG values) as the difference between the value at location “b” and the value at location “a”. Yet further, widths (values) that depend on the location of pairwise combinations of points may be computed (e.g., f(b,c)=width(b)/width(c)).

The tenth category of features that may be extracted from signal data representing a PPG waveform by the first ML process in step 106 (and step 209) may be referred to as “derivative waveform” features.

In an embodiment, a derivative waveform feature may be computed by generating a value or curve representing a first derivative and second derivative of a PPG waveform whose derivative has previously been computed as described above. Said another way, in an embodiment, each of the computations described above relating to an extracted feature may be repeated and then a derivative of each computation may be completed.

Turning to the second ML process, as noted previously, the second ML process may be viewed as related to the first ML process, except that instead of extracting features and generating values based on a large number of data points representing a PPG waveform, the features are extracted and the values are generated based on selecting certain points on the PPG waveform that are equidistant from one another. This is believed to reduce the computational complexity of the second ML process as compared to the first ML process. More particularly, as it pertains to the extracting of features described above, such features are identified based on selecting points in FIG. 3 that are equidistant from each other on the waveform rather than select, for example, the location of the systolic peak, dicrotic notch, and diastolic peak respectively.

Accordingly, with this distinction in mind, and to avoid being overly repetitious, in embodiments the second ML process may extract similar categories of features as the first ML process described above and generate related values.

In comparison to the first and second ML processes, when the AI process is incorporated into the referential process or method 100 such a process may include convolutional neural network processing or recurrent neural network processing or other neural network based or equivalent processes (e.g., algorithms) to implicitly extract the categories of features of a PPG waveform or waveform components described above to generate values that can be correlated to detectable patterns of physiological states. Optionally, the AI process may utilize values from the waveform decomposition, shown in step 108 in FIG. 1 (and step 211 in FIG. 2), that resulted in the same equidistant points on the PPG waveform that are equidistant from one another as with the second ML process (step 107 in FIG. 1, and step 210 in FIG. 2).

Continuing, upon completion of the first ML process, second ML process or AI process (or optionally prior to such a process) the so generated values (or if done prior to a process, the signal data) may be optionally, further transformed by the one or more electronic processors 2a to 2n by applying yet additional electronic filtering and trimming to, for example, remove values (or signal data) that are determined to be historically abnormal over a predetermined time period, and/or that is determined to be malformed based on a comparison with previous, historical physiological values. In more detail, for example, in optional step 109 values output by the first ML process. the second ML process or AI process may be compared (using processors 2a to 2n, for example) to values representing known, valid waveforms through use of additional ML or AI processes.

As an example, prior to executing step 109 the one or more processors 2a to 2n may be operable to generate reference, mean and standard deviation for each feature, or groups of features like the entire heart beat waveform, for all waveforms computed from step 105 for all subjects in a PPG data set from devices 1a to 1n and later stored for reference (the later data storage devices are not shown in FIG. 1 or 2).

In an embodiment, the evaluation as to whether signal data representing a waveform is valid in step 109 (or step 212) comprises comparing each waveform feature to the generated, reference mean and standard deviations (e.g., by using processors 2a to 2n, for example), In an embodiment, if the comparison indicates that the generated mean or standard deviation are outside a specified multiplier of the standard deviation then the signa dat representing a waveform is rejected. Said another way, the processors 2a to 2n (and 6a to 6n) may electronically exclude corrupted signals within the received electronic signals for an associated time segment (during which the corrupted signals were received), when one or more extracted features of the received electronic signals and/or an entirety of the received electronic signals is outside an acceptable range as compared to previously observed electronic signals of the same type within a past finite time period stored in an electronic database.

In an embodiment, the value of the multiplier value may be set based on the feature type, a related statistical distribution, and the desired tolerance for a type of waveform. For example, a lower value for the multiplier (of the standard deviation) may result in higher signal data (i.e., waveform) rejection rates and a “cleaner” set of signal data (waveforms), although this may result in waveforms being rejected that should have been accepted. In contrast, a higher value for the multiplier (e.g., 2 or 3 times the standard deviation) may result in a statistical acceptance of 97.7% or 99.9% of signal data representing waveform features, respectively, which in some situations may be too high of an acceptance rate. A similar process of earlier calculation of reference statistics and later comparison in step 109 (or step 212) would also occur for all features. Furthermore, ML processes such as K-means or K-mediods Clustering could be implemented by processors 2a to 2n (and 4a to 4n) to generate K>1 clusters, using feature calculations, for PPG signal data from storage devices 1a to 1n which could then be individually utilized to generate “k” possible mean values, standard deviation values, and multiplier(s) of standard deviation values. When determining if signal data representing a waveform is valid in step 109 (or step 212), each waveform feature may be compared to the “k” reference mean values and corresponding standard deviation (using processors 2a to 2n, or 4a to 4n). In an embodiment, if the comparison indicates that the value of the waveorm feature is outside of all specified corresponding multipliers of the corresponding standard deviation then the signal data representing a waveform is rejected.

For the reader's benefit, as noted above the first ML process, second ML process and AI process in steps 106 to 108 comprise electronic signal processing processes. In contrast, the ML and AI processes that may be completed during step 109 may comprise electronic filtering processes.

Continuing, during step 109 values output from step 106 to 108 may be adjusted (i.e., skewed, scaled, rotated, or otherwise transformed) to improve the representation of the original heart waveform signals and resulting values generated by method 100 (and method 200). Furthermore, for example, the values based on extracted waveform features may be compared to known, historical values of valid waveform features for an individual and/or general population in step 109 or later on during steps 110 to 113 (e.g., compared to physiological characteristics including ethnicity, age, sex, or other attributes or personalization data). Said another way, step 109 might consider that the input PPG waveform may have been slightly adjusted due to minor deficiencies in the sensor, algorithm inefficiencies in Signal Heartbeat Decomposition in step 105, or due to body response in presence of stimuli and may not exactly match previously seen PPG waveforms in storage device 1a. In another way, step 110 to 113 may consider that such adjustments are inherent to the PPG waveform and the PPG waveforms in storage device 1a are probably a subset of all possible PPG waveforms, and thus it may be prudent to also consider and further use, in steps 110 to 113, slightly adjusted PPG waveforms from a given PPG waveform in an appropriate fashion.

Before continuing further, it should be noted that the inventors believe that the extraction of the combination of PPG waveform features described above is innovative. Said another way, rather than extract 3,000 or more features from a PPG waveform the inventors have described categories of features and specific examples of such categories that can be explicitly extracted to more effectively correlate measured heart waveform signals (e.g., PPG signals) to a plurality of relative physiological states and levels and to specific physiological states and levels, such as hydration and heat stress states and levels.

Further, the inventors believe that the extraction of the combination of PPG waveform features described above based on the selection of certain points on a PPG waveform that are equidistant from one another is also innovative. Said another way, rather than extract 3,000 or more features from a PPG waveform the inventors have described categories of features and specific examples of such categories that can be extracted based on the selection of certain points on a PPG waveform that are equidistant from one another, thus reducing the computational complexity of analyzing a PPG waveform.

Yet further, the inventors believe that the implicit extraction of the combination of PPG waveform features and/or characteristics described above is innovative (using th ML2 or AI processes). Said another way, rather than extract 3,000 or more features from a PPG waveform the inventors have described categories of features and specific examples of such categories that can be implicitly extracted to more effectively correlate measured heart waveform signals (e.g., PPG signals) by the AI process.

Continuing, upon completion of one or more of steps 101 to 109 the resulting values representative of characteristics of heart waveforms (e.g., PPG waveforms) may be output to steps 110 to 113 for correlation to known, selected physiological states (e.g., known hydration and heat stress values at varying levels).

In embodiments, steps 110 to 113 may be completed by the execution of stored electronic instructions in memories of the one or more electronic processors 2a to 2n.

In more detail, in steps 110 to 112 values representing characteristics of heart waveforms (e.g., PPG waveforms) from previous steps 101 to 109 may be correlated to hydration and heat stress values and/or patterns that processors 2a to 2n previously received (and stored in memories not shown) from storage components 1b to 1n during step 101a that indicate one or more physiological states (e.g., hydration and heat stress values at varying levels and body position).

For example, one physiological state is dehydration that, for example, may be measured by determining the water loss of an individual. Another physiological state is heat stress that, for example, may be measured by determining the core body temperature(s) or skin temperature(s) of an individual. The relationship between the values output from previous steps in method 100 to stored hydration and heat stress values/patterns may indicate a relative physiological state (e.g., dehydration state or heat stress state).

In more detail, in embodiments a plurality of signal data based values from one or more of steps 101 to 109 may be input into step 110. During step 110 each of the plurality of values may be correlated to historical hydration states and patterns that have been previously mapped to heart waveform data (PPG data) to determine a relative indication of the hydration state of the inputted signal data. In embodiments, step 110 may comprise identifying the relative, hydration states that most closely correlate with each of the the plurality of inputted values.

For example, referential data related to hydration states from step 101a may comprise specific values, for example, values indicating a percentage(s) fluid loss when an individual is at different stages of of an experiment after periods of fluid loss (including when fully hydrated which is equivalent to 0% fluid loss) due to exercise and/or exposure to ambient heat (to induce sweat). In an embodiment, these values may then be correlated during step 110 to signal data representing waveforms captured at each of these hydration stages from steps 101 to 109, using either a ML or AI process.

For example, hydration state data from step 101a may be specific values of percentage fluid loss when a subject is at different stages after periods of fluid loss (including when fully hydrated which is equivalent to 0% fluid loss) due to exercise and/or exposure to ambient heat to induce sweat, and these values are then correlated, in step 110, with waveforms captured at each of these stages from steps 101 to 109, using either a machine learning algorithm or artificial intelligence algorithm. When utilizing ML Approach 1, feature extracted values of the waveforms are used with a machine learning algorithm in step 110, such as but not limited to Random Forest or other classifier algorithm. When utilizing ML Approach 2, the equidistant waveform values are used with a machine learning algorithm in step 110, where the algorithm may be one of the same as with MLApproach 1. Whereas when utilizing an AI approach, either the equidistant waveform values or the raw waveform values may be used with a neural network based algorithm in step 110, such as but not limited to CNN or RNN algorithms.

The associated plurality of inputted signal values and the correlated hydration states and their relationships to one another may be stored in storage device 7a to 7n (where “n” represents the last storage device) in step 114 and may form a reference “hydration index model”.

Similarly, in embodiments a plurality of signal data based values from one or more of steps 101 to 109 may be input into step 111. During step 111 each of the plurality of values may be correlated to historical, relative heat stress states and patterns that have been previously mapped to heart waveform data (e.g., PPG data) to determine a relative indication of the heat stress state of the inputted signal data. In embodiments, step 111 may comprise identifying the relative historical heat stress states that most closely correlate with each of the the plurality of inputted values.

In embodiments, the correlated heat stress states may be further processed to determine one or more heat stress states, including, but not limited to, determining an estimated core body temperature or skin temperature in degrees Fahrenheit or Celsius.

In more detail, referential data related to heat stress from step 101a may be correlated during step 111 to signal data representing waveforms captured at each heat stress stage from steps 101 to 109, using either a ML or AI process.

For example, heat stress state data from step 101a may be specific values of core body temperature or skin temperature in degrees Fahrenheit or Celsius when a subject is at different stages after periods of body temperature increases due to exercise in various ambient temperatures and/or exposure to dehydration to induce sweat, and these values are then correlated, in step 111, with waveforms captured at each of these stages from steps 101 to 109, using either a machine learning algorithm or artificial intelligence algorithm. When utilizing ML Approach 1, feature extracted values of the waveforms are used with a machine learning algorithm in step 110-111, such as but not limited to Random Forest or other classifier algorithm. When utilizing ML Approach 2, the equidistant waveform values are used with a machine learning algorithm in step 111, where the algorithm may be one of the same as with ML Approach 1. Whereas when utilizing an AI approach, either the equidistant waveform values or the raw waveform values may be used with a neural network based algorithm in step 111, such as but not limited to CNN or RNN algorithms.

The correlated historical heat stress states and the associated plurality of inputted signal values and their relationships to one another may be stored in storage device 7a to 7n in step 115 and may form a reference “heat stress index model”.

For the reader's reference, it should be noted that the hydration index model and heat stress index model may be based on values that were generated by explicit or implicit extraction of heart waveform features (e.g., PPG waveform features).

In addition to the formation of hydration index and heat stress index models, in an embodiment a combined (inter-related) hydration and heat stress index process (i.e., model) may be formed in step 112. The hydration index model captures the correlation between signal data representing heart waveforms and dehydration states, and the heat index model captures the correlation between signal data representing heart waveforms and heat stress states, whereas the combined hydration and heat stress model captures—in a single model—the correlation between signal data representing the heart waveforms and together the physiological impact due to both dehydration and heat stress together.

For example, when a person is exercising in a very hot environment a combined hydration and heat stress index may indicate physiological distress, whereas the separate hydration index or heat index will be able to distinguish what specifically is the underlying issue, whether the person is dehydrated, or under heat stress, or both.

In more detail, the combined hydration and heat stress index (HHI index) may, as an example, be represented logically by the process:

HHI_index=(α*heat+β*hydr+δ*heat*hydr)

where the HHI_index is defined as a function of the combination of heat (e.g., heat stress), hydration (i.e. dehydration), and combination heat and hydration levels. Specifically, heat and hydr are estimated heat and hydration measures, computed internally in a means similar to the computation of the hydration index and heat index above, or provided as inputs. α, β and δ are estimated coefficients computed during a machine learning or artificial intelligence process.

During step 112 a plurality of signal data based values from one or more of steps 101 to 109 may be input into step 112. Each of the plurality of values may be correlated to historical hydration states and patterns and, in addition, to historical heat stress states and patterns that have been previously mapped to heart waveform data (PPG data) to substantially simultaneously determine a relative indication of the combined effect of hydration and heat stress states of the inputted signal data. In embodiments, step 112 may comprise substantially simultaneously identifying the relative, hydration and heat stress states that most closely correlate with each of the the plurality of inputted values.

In embodiments, the stored correlated hydration and heat stress states may be further processed to determine a combined hydration and heat stress state or states, for example.

For example, hydration state data from step 101a may be specific values of percentage fluid loss when a subject is at different stages after periods of fluid loss (including when fully hydrated which is equivalent to 0% fluid loss) due to exercise and/or exposure to ambient heat to induce sweat, and heat stress state data from step 101a may be specific values of core body temperature or skin temperature in degrees Fahrenheit or Celsius when a subject is at different stages after periods of body temperature increases due to exercise in various ambient temperatures and/or exposure to dehydration to induce sweat, and these values are then correlated, in step 112, with waveforms captured at each of these stages from steps 101 to 109, using either a machine learning algorithm or artificial intelligence algorithm. When utilizing ML Approach 1, feature extracted values of the waveforms are used with a machine learning algorithm in step 112, such as but not limited to Random Forest or other classifier algorithm. When utilizing ML Approach 2, the equidistant waveform values are used with a machine learning algorithm in step 112, where the algorithm may be one of the same as with ML Approach 1. Whereas when utilizing an AI approach, either the equidistant waveform values or the raw waveform values may be used with a neural network based algorithm in step 112, such as but not limited to CNN or RNN algorithms.

The inventors believe that the ability to determine a combined (inter-related) hydration and heat stress index and related states associated with PPG waveforms, and their interrelationship to one another (as described herein) is innovative.

The correlated hydration and heat stress indices, states, the associated plurality of inputted signal values and their relationships to one another may be stored in storage device 7a to 7n in step 116 and may form a reference “hydration and heat index model”.

Similarly, additional models may be formed. For example, a hydration index position model, heat stress index position model and a combined hydration and heat stress index position model may be formed and stored in storage devices 7a to 7n based on (i) the same plurality of signal data based values from one or more of steps 101 to 109, (ii) the correlation of such values to either referential historical hydration, heat stress or a combination of hydration and heat stress states and patterns from storage devices 1a to 1n and, in addition, positional states and patterns that have been previously mapped to heart waveform data (PPG data)(collectively “stored historical values”). In embodiments, the states that most closely correlate with each of the the plurality of inputted values may be determined.

For example, waveform data (e.g., PPG) may be collected for subjects in different physical positions, including sitting, standing, or supine using data from signal data steps 101 to 109 and/or stored referential data from step 101a, and correlated with the dehydration, heat stress, and combination, for the purpose of creating a hydration index position model, a heat index position model, and/or a hydration and heat index position model.

The correlated physiological states, the associated plurality of inputted signal values and their relationships to one another may be stored in storage device 7a to 7n as respective models as a part of one or more of steps 114 to 116.

Method 100 may also comprise the formation of a combined hydration and heat stress interpretative model in step 113. Such a model may provide absolute hydration and/or heat stress values (e.g., how many millimeters of water should an individual drink after exercising?) as compared to providing a correlation to relative hydration index, heat stress index, and/or hydration and heat stress index.

For example, in an embodiment the one or more processors 2a to 2n may be operable to electronically convert the hydration index, heat stress index, and/or hydration and heat stress index to absolute hydration and temperature related values that are more meaningful to an individual.

While the generation of index values may be used to indicate relative hydration and temperature values, to generate absolute hydration and temperature values only the signal data representing heart waveforms may be required (at a minimum) to generate the absolute values.

In an embodiment, a plurality of signal data based values from one or more of steps 101 to 109 may be input into step 113. Each of the plurality of values may be correlated to (i) historical hydration states and patterns, and historical heat stress states and patterns that have been previously mapped to heart waveform data (PPG data) and (ii) personalized hydration and heat stress values to substantially simultaneously determine an absolute hydration and/or heat stress level as well as information regarding how to achieve an absolute hydration or heat stress level. In embodiments, step 113 may comprise identifying an absolute hydration and/or heat stress level or amount (e.g., how many millimeters of water should an individual drink after exercising; and/or % dehydrated) that most closely correlate with each of the the plurality of inputted values.

In more detail, the one or more processors 2a to 2n may be operable to generate absolute values by implementing an interpretative process that may be expressed as a function of the hydration index model (processes), the heat index model (processes), and personalization values, such as:

wtloss or temp=subject_{δ}*(α*heat+β*hydr+γ*heat*hydr)

where subjects are values from a biographical process (not shown in FIG. 1) that captures personalization data of an individual or individuals, heat and hydr are heat stress and hydration index values, respectively, generated from signal data, and α β γ are parameters determined during a machine learning process or artificial intelligence process and are common to all subjects (i.e. not subject specific). subject_{δ} are parameters that are subject specific, such as, for example, sex, height, and fitness level. Personalization data may include sex, height, mass, and body fat, as well as hydration and heat tolerance measures.

In even further detail, the interpretative process of step 113 may depend on both the processes and values included in the heat index model and the hydration index model, or may depend on the processes and values of the combination hydration and heat model, but in any case will include computed components representing the hydration level and heat level of a person. In addition, the interpretative process may utilize personalization values that are individualized indicators of heat stress and/or dehydration tolerance, although these additional personalization values are optional. While the interpretative process may generate absolute hydration and temperature values without personalization data, the accuracy of the values is believed to be improved by including personalization data.

As described above, step 113 comprises the process of correlating heat and hydration states, and personalization data, to absolute hydration (e.g., how many millimeters of water should an individual drink after exercising?) and/or heat (e.g., core body temperature) values, and, for example. Such a correlation process may comprise a Bayesian statistical process that includes values of the heat index, hydration index, and personalization data.

The inventors believe that the ability to identify and generate absolute hydration and/or heat stress values (levels), rather than relative physiological states, is innovative.

The correlated hydration and heat stress states, the personalized values, along with the associated plurality of inputted signal values and their relationships to one another may be stored in storage device 7a to 7n in step 117 and may form a reference “hydration and heat stress interpretative model”.

Relatedly, a hydration, heat stress and position interpretative model may be formed during step 117.

In an embodiment, the same plurality of signal data based values from one or more of steps 101 to 109 may be input during step 117 and then correlated to (i) a combination of hydration and heat stress states and patterns, and positional states and patterns that have been previously mapped to heart waveform data (PPG data)(collectively “stored historical values”) as well as (ii) personalization data. In embodiments, the states and personalization data that most closely correlate with each of the the plurality of inputted values may be determined.

The correlated states, personalization data, the associated plurality of inputted signal values and their relationships to one another may be stored in storage device 7a to 7n in step 117 and may form a reference “hydration, heat stress and position interpretative model”.

Again, it should be noted that all of the models described above may be based on values that were generated by explicit or implicit extraction of heart waveform features (e.g., PPG waveform features).

Thereafter, the stored models may be used to estimate current, real-time hydration and heat-stress states, among other things, and predict future hydration and heat-stress values for a current individual in method 200 described below in order to provide medical attention, if necessary (e.g., life-saving medical attention) or for personal monitoring and physical performance improvement. Said another way, as explained further below, one or more electronic processors, that may be made a component(s) of a monitoring device (e.g., mobile device, wrist watch, wrist band), may be configured to transform current (as opposed to historical) measured, heart waveform signals to values representing hydration and heat stress levels and predict future, estimated values of hydration and heat-stress levels of a current individual (e.g., an individual currently involved in an athletic activity or exercise routine, an injured individual, or a patient in a hospital) based, in part, on the models discussed above.

It should be understood that while FIG. 1 depicts four separate steps 110, 111, 112, and/or 113 and eight separate memories 7a, 7b, 7c, and/or 7n this is merely exemplary. Alternatively, the models may be stored using fewer processing steps and in fewer memories or may be stored using more processing steps and in more memories depending on, for example, the amount of physiological data, the number different physiological states and/or the size/processing speed of the electronic memories needed to store the correlated values.

Referring now to FIG. 2 there is depicted a simplified block diagram of an innovative real-time process or method 200 for estimating current and real-time hydration and heat stress states and levels of a current individual and predicting future, estimated hydration and heat stress states and levels for such an individual. The inventors believe that such estimates may be critical in life-saving efforts as well as in personal, physiological monitoring and physical performance improvement.

In an embodiment, copies of some or all of the electronic instructions and values (e.g., data) stored in the electronic memories of processors 2a to 2n or other components involved in method 100 may be electronically transferred to one or more electronic processors 4a to 4n or other components involved in method 200, where processors 4a to 4n may be a component(s) of a user's device (e.g., a device used by an individual interested in personal, physiological monitoring such as a watch, mobile phone, laptop computer or PC or a device that may be used by a medical institution, hospital, professional, technician or attendant, such as a server, PC). Alternatively, the instructions and values may be stored on processors 4a to 4n using other existing electronic means and processes.

In addition, electronic signals representing one or more of the models stored in one or more of the storage devices 7a to 7n may be electronically transferred from one or more of the storage devices 7a to 7n to one or more of the electronic processors 4a to 4n or other components involved in method 200. In the embodiment depicted in FIG. 2, the models may be transferred to one or more processors labeled 4b.

Similar to the referential method 100 described herein, the one or more processors 4a to 4n may receive a plurality of current signal data (as compared with referential, historical signal data from storage devices 1a to 1n) representative of heart waveform signals from sensors 3a to 3n (e.g., PPG data, ECG data and other heart waveform signal data described herein) in steps 201 to 203, and, optionally, may receive current attribute and personalization data of an individual from memory 3aa in step 204 (collectively referred to as “personalization data”).

Upon receiving such data the one or more electronic processors 4a to 4n executing method 200 may be operable to retrieve instructions stored in their electronic memory (not shown) in order to transform all of the the received data (collectively referred to as “current data”) into values representing current hydration, heat stress states and levels or some combination of hydration and heat stress states and levels (e.g., a hydration and heat stress index) using additional steps described herein.

In more detail, as an option, the one or more electronic processors 4a to 4n may be operable to transform the received current data into an improved representation of such data by electronically removing unwanted or undesirable electronic noise or artifacts in step 205 as described previously to increase the accuracy of the received data. In addition, as another option, the one or more electronic processors 4a to 4n may be further operable to transform the data received from sensors 3a to 3n by electronically applying position and/or motion adjustment steps 206, 207 (similar to steps 103,104 described with respect to method 100 in FIG. 1) in order to adjust the current data to compensate for such different speeds, or other causes of motion related noise, and positions made by the user that is connected to sensors 3a to 3n, for example.

Continuing, upon completing one or more of optional steps 205 to 207, the current signal data may then be further transformed by decomposing it into one or more different, constituent data sets representing constituent heart waveform signals (e.g., PPG waveforms, ECG waveforms) or waveform components in step 208 as described above with respect to step 105.

As before, for ease of explanation we will select PPG waveform signals as the signals of interest though, again, this is merely exemplary and non-limiting (i.e., the signal data can be different than PPG signal data, e.g., ECG signal data).

During step 208 the one or more electronic processors 4a to 4n may be operable to identify constituent PPG waveform components within the current data. Such data (or data segments) may, for example, represent a period of time corresponding to the heartbeat of an individual (e.g., data indicative of the start and end periods of the heartbeat) and may include past heartbeat detection information retrieved from an electronic memory or storage component (not shown) for predicting the likely time occurrence of a subsequent heartbeat.

The current signal data may be corrupted which may prevent the one or more electronic processors 4a to 4n from clearly identifying a representative PPG heart waveform or waveform component. Accordingly, in such an instance the processors 4a to 4n may (optionally) execute stored instructions in their electronic memories to exclude such corrupted data from further processing for an associated time segment. Accordingly, this function of the processors 4a to 4n may effectively act as an electronic filter of corrupted data received over heartbeat time segments. Upon completion of optional step 208, the resulting values representative of current physiological states may be output to steps 209 to 211 for further signal processing by the one or more electronic processors 4a to 4n depending on the ML or AI approach selected.

In embodiments, the one or more processors 4a to 4n may execute electronic signals stored as instructions in their electronic memories (not shown) to complete one or more of the processes in steps 209, 210 and/or 211 in FIG. 2. Said another way, a device (e.g., mobile device, PC, server) may be programmed to include instructions to execute the process in step 209, 210 or 211 or some combination of steps 209, 210 and 211.

To avoid being overly repetitious, in embodiments, steps 209 to 211 in method 200 comprise substantially the same steps as steps 106 to 108 in method 100, except, of course that steps 209 to 211 involve current data. Accordingly, subject to the proviso just stated, the description of steps 106 to 108 above is incorporated herein with respect to steps 209 to 211.

As before, the exemplary first ML process completed during step 209 may be used when it is desirable to know, and trace, the type of features that have been extracted from a heart waveform (i.e., traceability), while the exemplary second ML process completed during step 210 may be used when traceability is not a concern. In embodiments, completing the second ML process to implicitly extract features in order to generate values may require less computational energy for processors 4a to 4n or any associated device as compared to the first ML process (e.g., less power, and perhaps lead to longer battery life). Further, the exemplary AI process completed during step 211 may require the most computational energy to complete its processes and, thus, may be attractive when a substantial amount of current signal data is available to process (e.g., by a hospital). Similar to steps 106 to 108, it should be understood that each of the first ML process, second ML process and AI process in steps 209 to 211 comprise electronic signal processing processes. Similar to step 108, step 211 of the AI process includes convolutional neural network processing or recurrent neural network processing or other neural network based or equivalent processes (e.g., algorithms) to transform the current signal data into detectable patterns of physiological data.

As noted previously, during step 211 the AI process (and ML 2 process) may implicitly extract features of a heart waveform signal and/or waveform components that may be correlated to a heat or hydration state or value.

Continuing, the first ML process in step 209 may identify and generate values that correspond to certain heart waveform features, for example PPG waveform features. In embodiments (and as explained further below in the EXPERIMENTS section) the one or more electronic processors 4a to 4n may identify over 3,000 features of a single PPG waveform (“Current, Raw PPG waveform data”). Upon identifying and computing the values of the Current Raw PPG waveform data the first ML process may be further operable to transform such data into “Current, Normalized PPG waveform data” (e.g., a unit of measurement between 0 and 1). As explained previously above, by transforming (or converting) the Current, Raw PPG waveform data into Current, Normalized PPG waveform data inconsistencies inherent in the data are minimized and errors are reduced.

As stated before, the inventors believe that the transformation or conversion of measured PPG waveforms to a normalized unit of measurement generates more consistent predictions (i.e., predictive values) especially when current PPG waveforms may be sampled at different sampling rates (e.g., 25 Hz versus 75 Hz) and are of different lengths due to varying heart rates.

In embodiments, each of the categories of features described with respect to step 106 may also be extracted during step 209 and the resulting values stored by the one or more processors 4a to 4n. Similarly, the second ML process completed during step 210 and the AI process completed during step 211 comprise similar steps as completed during steps 107 and 108 described previously; with the proviso that steps 209 to 211 involve processing current data.

As noted previously, the second ML process completed during step 210 may be viewed as related to the first ML process completed during step 209, except that instead of extracting features and generating values based on a large number of data points representing a PPG waveform, the features are extracted and the values are generated based on selecting certain points on the PPG waveform that are equidistant from one another. Again, this is believed to reduce the computational complexity of the second ML process as compared to the first ML process. More particularly, such features may be identified based on selecting points from a PPG waveform that are equidistant from each other rather than select, for example, the location of the systolic peak, dicrotic notch, and diastolic peak respectively.

Accordingly, with that distinction in mind, and to avoid being overly repetitious, in embodiments the second ML process in step 210 may extract similar categories of features as the first ML process in step 209 and generate related values.

In comparison to the first and second ML processes, when the AI process is incorporated into the method 200 such a process may include convolutional neural network processing or recurrent neural network processing or other neural network based or equivalent processes (e.g., algorithms) to implicitly extract the categories of features of a PPG waveform or waveform components described above to generate values that can be correlated to detectable patterns of physiological states.

Continuing, upon completion of the first ML process, second ML process or AI process (or optionally prior to such a process) the so generated values (or if done prior to a process, the current signal data) may be optionally, further transformed by the one or more electronic processors 4a to 4n by applying yet additional electronic filtering and trimming to, for example, remove values (or signal data) that are determined to be historically abnormal over a predetermined time period, and/or that is determined to be malformed based on a comparison with previous, historical physiological values. In more detail, for example, in optional step 212 values output by the first ML process. the second ML process or AI process may be compared to values representing known, valid waveforms through use of additional ML or AI processes.

For the reader's benefit, as noted above the first ML process, second ML process and AI process in steps 209 to 211 comprise electronic signal processing processes. In contrast, the ML and AI processes that may be completed during step 212 may comprise electronic filtering processes.

Continuing, during step 212 values output from step 209 to 211 (and step 109) may be adjusted (i.e., skewed, scaled, rotated, or otherwise transformed) to improve the representation of the original heart waveform signals and resulting values generated by method 200. Furthermore, for example, the values based on extracted waveform features may be compared to known, historical values of valid waveform features for an individual and/or general population in step 212 or later on during steps 213 to 216 (e.g., compared to physiological characteristics including ethnicity, age, sex, or other attributes or personalization data).

Before going further, it again should be noted that the inventors believe that the extraction of the combination of PPG waveform features in method 200 is innovative. For example, rather than extract 3,000 or more features from a PPG waveform the inventors have described categories of features and specific examples of such categories that can be explicitly extracted to more effectively correlate current, measured heart waveform signals (e.g., PPG signals) to a plurality of physiological states and levels, such as hydration and heat stress states and levels.

Further, the inventors believe that the extraction of the combination of PPG waveform features in method 200 based on the selection of certain points on a PPG waveform that are equidistant from one another is also innovative. Said another way, rather than extract 3,000 or more features from a PPG waveform the inventors have described categories of features and specific examples of such categories that can be extracted based on the selection of certain points on a PPG waveform that are equidistant from one another, thus reducing the computational complexity of analyzing a PPG waveform.

Still further, the inventors believe that the implicit extraction of the combination of PPG waveform features in method 200 is innovative. Said another way, rather than extract 3,000 or more features from a PPG waveform the inventors have described categories of features and specific examples of such categories that can be implicitly extracted to more effectively correlate measured heart waveform signals (e.g., PPG signals) by the ML2 or AI process.

Continuing, upon completion of one or more of steps 201 to 212 the resulting values representative of characteristics of heart waveforms (e.g., PPG waveforms) may be output to steps 213 to 217 for correlation to known, selected physiological states (e.g., known hydration and heat stress values at varying levels).

In an embodiment, during optional step 213 the one or more processors 4a to 4n may be operable to adjust the values output from previous steps based on a position of an individual who is utilizing method 200 via a device. In embodiments the position of the individual may (a) be detected by one or more of the sensors 3a to 3n, (b) be derived by interpretation of data from the one or more sensors 3a to 3n, and/or (c) specified by the individual.

In more detail, corresponding values from previous steps may be adjusted in step 213 by, for example, applying a predetermined set of adjustment factors that are position-dependent or, optionally, by applying adjustment factors (i.e., values) that may have been based on previously determined values for a given position derived through a separate machine learning or artificial intelligence process (such separate ML and AI steps are not shown in FIG. 2).

Upon completion of one or more of steps 201 to 213 the resulting values representative of characteristics of current heart waveforms (e.g., PPG waveforms) may be output to steps 214 to 217 for correlation to one or more of the physiological models stored in one or more processors 4a to 4n (e.g., processor 4b).

In more detail, in steps 214 to 216 the plurality of values representing characteristics of current heart waveforms (e.g., PPG waveforms) from previous steps 201 to 213 may be correlated to models developed by method 100.

For example, during step 214 each of the plurality of values may be correlated to the hydration index model to determine a relative indication of current hydration levels and states associated with the inputted, current signal data. In embodiments, step 214 may comprise identifying the relative indication(s) of current hydration levels and states that most closely correlate with each of the the plurality of inputted values.

In more detail, an exemplary hydration index resulting from the correlation process described herein may be represented by a value in the range of values from 0 to 1, where the value “0” represents an euhydrated (fully hydrated) state, and the value “1” represents a significantly dehydrated state. While the exemplary hydration index may be represented by a value between 0 and 1 this is merely exemplary. Alternatively, the hydration index resulting from the correlation process discussed herein may be represented other values other than 0 to 1.

In embodiments, the correlated, relative current hydration levels and states may be further processed to determine one or more current, hydration levels and states, including, but not limited to, determining a current hydration level or state as percentage of body mass or volume of fluid change, and/or to determining a current hydration level or state based on a change in the amount of bodily fluids, whether up or down, for example, that may indicate a current hydration (or dehydration) level or state.

The associated plurality of inputted signal values and the correlated current hydration levels and states and their relationships to one another may be stored in storage device 5a to 5n (e.g., devices 5a and 5d, where “n” represents the last storage device) in step 218.

Similarly, in embodiments a plurality of signal data based values from one or more of steps 201 to 213 may be input into step 215. During step 215 each of the plurality of values may be correlated to the heat stress model to determine relative indications of current heat stress levels and states of the current signal data. In embodiments, step 215 may comprise identifying the current, relative heat stress levels and states that most closely correlate with each of the the plurality of inputted values.

In more detail, an exemplary heat stress index resulting from the correlation process discussed herein may be represented by values in the range of 0 to 1, where the value of “0” represents a normal body temperature, and the value of “1” represents a significantly elevated body temperature, where the body temperature may be a core body temperature or a skin temperature, for example.

While the exemplary heat stress index may be represented by a value between 0 and 1 this is merely exemplary. Alternatively, the heat stress index resulting from the correlation process discussed herein may be represented other values other than 0 to 1.

In embodiments, the correlated, current heat stress levels and states may be further processed to determine one or more current heat stress levels and states, including, but not limited to, determining current, estimated core body temperatures or current, skin temperatures in degrees Fahrenheit or Celsius.

The correlated, current heat stress levels and states and the associated plurality of inputted signal values and their relationships to one another may be stored in storage devices 5a to 5n in step 218 (e.g., devices 5b and 5n).

In addition to separately correlating hydration and heat stress levels and states, in an embodiment, combined (inter-related) hydration and heat stress levels and states may be correlated in step 216.

During step 216 a plurality of current signal data based values from one or more of steps 201 to 213 may be input into the combined hydration and heat stress index model. Each of the plurality of values may be correlated to the hydration levels and states and, in addition, to heat stress levels and states to substantially simultaneously, or independently, determine a current, relative indication of hydration and heat stress levels and states of the inputted signal data. In embodiments, step 216 may comprise substantially simultaneously identifying the relative, hydration and heat stress levels and states that most closely correlate with each of the the plurality of inputted values.

In more detail, an exemplary, combined hydration and heat index resulting from the correlation process described herein may be represented by values in the range of 0 to 1, where a value of “0” represents a normal body temperature and a normal hydration level, and a value of “1” represents a significantly elevated body temperature, where the body temperature may be a core body temperature or a skin temperature, and/or a significantly dehydrated state.

While the exemplary, combined hydration and heat stress index may be represented by a value between 0 and 1 this is merely exemplary. Alternatively, the combined hydration and heat stress index resulting from the correlation process discussed herein may be represented other values other than 0 to 1.

In embodiments, the correlated, current hydration and heat stress levels and states may be further processed to determine combined hydration and heat stress levels and states, for example.

The inventors believe that the ability to determine combined (inter-related) hydration and heat stress levels and states associated with current PPG waveforms, and their interrelationship to one another (as described herein) is innovative.

The correlated, current hydration and heat stress levels and states, the associated plurality of inputted signal values and their relationships to one another may be stored in storage device 5a to 5n in step 218 (e.g., device 5c).

Again, it should be noted that all of the current hydration and heat stress values and states described above may be based on values that were generated by explicit or implicit extraction of current heart waveform features (e.g., PPG waveform features) correlated to referential data, including data the related to individuals placed in different positions (e.g., standing, sitting, supine)(i.e., position or positional data).

The processes implemented during steps 110-113 of method 100 may generate index values that had also been correlated to referential positional data, and as such the hydration index position model, heat index position model, hydration and heat index position model, and/or the interpretative position models that are stored in one or more processors 4a to 4n (e.g., processor 4b) may be implemented during step 213 when positional data is available and is to be considered.

Method 200 may also comprise a comparison step 219. In an embodiment, the current hydration and heat values and states may be compared to historical values and states, for example. In an embodiment, by completing such a comparison in step 219 the real-time method 200 may generate correlated hydration and heat stress levels and states that more accurately represent the current physiological state(s) of an individual(s).

The correlated current levels and states may also be used to predict future estimates of the user's hydration and heat-stress levels and states in step 220 using a predictive process that, in an embodiment, may compare current hydration and heat stress levels and states to stored, past hydration and heat stress levels and states from electronic data storage 6a and output predictive, estimated hydration and heat stress levels and states in step 222, for example, in order to provide medical attention to the individual or user, if necessary (e.g., life-saving medical attention) or for the user's personal, physiological monitoring to take preventive measures before medical attention becomes necessary. The predicted estimates may be stored in one or more electronic memories 8a to 8n, for example. In further detail, the one or more processors 4a to 4n may be operable to complete step 220, for example, by completing linear regression processes or more advanced ML and/or AI processes.

Step 220, may also produce recommended actions (e.g., “consume 100 ml of water” to rehydrate) for addressing the current or predicted hydration or heat level of the individual, where recommended actions may be stored in memories 9a to 9n for example in step 221.

It should be understood that while FIG. 2 depicts separate steps 201 to 222, and a number of separate storage devices or memories 5a to 5n, 6a, 8a to 8n, and 9a to 9n this is merely exemplary. Alternatively, levels and states representing a current or predicted physiological state(s) may be stored using fewer processing steps and in fewer storage devices and memories or may be stored using more processing steps and in more storage devices and memories shown in FIG. 2 depending on, for example, the amount of physiological data (current signal data), the number of different physiological types of data and/or the size/processing speed of the electronic storage devices or memories needed to store correlated levels and states.

In an embodiment, the values in memories 5a to 5n, 6a, 8a to 8n and/or 9a to 9n may be revised as more and more data is received and processed using steps 201 through 222.

Additionally, the real-time sensing method 200 (and corresponding processors 4a to 4n) may include the generation of one or more control signals to control the output of the values stored in memories 5a to 5n, 6a, 8a to 8n, and/or 9a to 9n (and/or one or more alarm indicators indicating one of the values has exceeded a threshold set by a user, or a preset recommended threshold) to one or more display devices (not shown in figures) that may be a component of a user device 200a. In embodiments, the control signals may be generated as a part of a separate step or as a part of step 214, 215, 216, and/or 217, for example.

Throughout the discussion above, and through experimentation, the inventors have observed that the features and shape of a PPG waveform, for example, may be affected by (1) the position (or positions) of an individual (e.g. standing, sitting and supine) and (2) changes in the position of an individuals limbs, such as raising or lowering one or more arms or legs which is caused by gravity forcing the redistribution of blood within the circulatory system (as the primary force driving blood through the body is hydrostatic pressure (blood pressure) from the heart pumping blood through the circulatory system).

A reflex typically referred to as the “baroreceptor reflex” is a mechanism that the human body takes to keep blood pressure in normal range due to abrupt changes in positions. Due to these mechanisms, the amplitude of the systolic and diastolic peaks change with the different positions.

Realizing this, the innovative methods and devices discussed herein account for such positions. For example, during step 103 of method 100 (and corresponding step 206 of method 200) the raw waveform signals based on the determined position of the individual (or a limb) may be an adjusted based on data received from one or more sensors, such as a gyroscope(s) and/or accelerometer(s). The overall amplitude of the signal, for example, may be adjusted up or down by a predetermined factor for the given position.

As an additional example, the one or more processors 2a to 2n may execute stored instructions retrieved from their memories (not shown) to complete a signal mapping process that maps (i.e., assigns) signal data representing one or more time-aligned segments of a waveform (e.g., PPG, ECG waveform) to one or more time-aligned segments of another waveform, from supine to standing or vice versa, as explained further in co-pending U.S. patent application Ser. No. ______, the contents of which are incorporated by reference herein as if set forth in full herein.

In an embodiment, the various models stored in devices 7a-7n (e.g., “heat index position model” and other position models) can be represented as some function f( ) of the data. e.g. model=f(data). An exemplary model that uses linear parameters can be represented as: model=a₁*feature₁+a₂*feature₂+ . . . +aₙ*featureₙ, where data=features (e.g.; feature₁, feature₂, . . . , featureₙ) are extracted from a waveform segment (PPG or ECG) using one or more of the aforementioned categories of features and parameters (a₁, a₂, . . . , aₙ) estimated by statistical processes such as a least squares regression process. Additionally, the position is implicitly or explicitly known, the exemplary model may be updated to include the position as:

model=(a₁ₘ*feature₁+a₂ₘ*feature₂+ . . . +aₙₘ*featureₙ)*(when position=supine)+(a₁ₖ*feature₁+a₂ₖ*feature₂+ . . . +aₙₖ*featureₙ)*(when position=standing)

where data=features (e.g.; feature₁, feature₂, . . . , featureₙ) are extracted from a waveform segment (e.g., PPG, ECG) using one or more of the aforementioned categories of features and parameters (a₁ₘ, a₂ₘ, . . . , aₙₘ) where parameters (a₁ₖ, a₂ₖ, . . . , aₙₖ) may be estimated by statistical processes (e.g., like least squares regression) using data that is limited to when individuals are in a supine position and when such individuals are in a standing position, respectively. The inventors believe that using position data within the formulation of the model parameters will improve the accuracy of the model by incorporating the slight changes in a waveforms shape due to changing body position. Yet further, by explicitly using position as a factor to influence the generation of parameters for the model, more accurate models can be estimated.

In the foregoing discussion it should be understood that aforementioned linear model (using linear parameters) is merely exemplary. Alternative processes may be used, such as an ML or AI process to estimate the parameters related to supine, standing, sitting, or other types of positions.

Yet further, steps 114 to 117 include additional processes that may be used to account for an individual's position, where, for example, PPG signals may be sensed for individuals at different positions at each of the states. In more detail and making use of the hydration index model, this model may process PPG waveform features or the waveforms themselves (depending on which process—ML1, ML2 or AI is implemented) in association with varying hydrated states (euhydrated to significantly dehydrated). The hydration index position model furthers this by varying the position at each state, thus the models are trained on PPG signals that include position and thus have been determined when used during evaluation in steps 214 to 217 to produce equivalent and accurate index values for different positions. It should be noted that position related signals need not be included in the models, thus the models stored in electronic storage devices 7a to 7n need not include positional data. a separate set of models can be developed that include position for 7a to 7n.

Experiments

Exemplary experiments were conducted that used one or both of processes/methods 100 and/or 200. Referential, hydration and heat stress related data was collected from a number of individuals and then stored in storage devices similar to devices 1a to 1n. To collect the hydration and heat stress related data, measurements were made during, and immediately following, periods of activity and inactivity for each individual. Measurements were also taken in ambient room conditions as well as in high temperature environments, with each individual in an euhydrated state (i.e., an individual's body had a normal amount of fluid) and a dehydrated state.

Specific experimental parameters and terminology:
“Baseline” state: room temperature (24 degrees Celsius and 55% relative humidity) of an euhydrated individual;
“TempH” state: room temperature while individual is euhydrated;
“TempD” state: room temperature, individual dehydrated (achieved by individual abstaining from fluids prior to test);
“EhH” state: individual euhydrated and remained euhydrated in a high-heat (35 degrees Celsius and 55% relative humidity) setting;
“EhD” state: individual dehydrated and remained dehydrated in a high-heat (35 degrees Celsius and 55% relative humidity) setting;

Each individual involved in the experiments performed strenuous exercises while subjected to each of the above-mentioned states. Each individual underwent continuously measurements using multiple physiological sensors, including a rectal body thermometer (used to measure core body temperature) and a PPG finger sensor. Personalized data was collected for each individual, including body weight, height, and sex. VO2Max tests were performed on each individual.

In accordance with exemplary method 100, noise and artifacts were removed (i.e., filtered out) from data representing PPG waveforms (step 102). The data representing PPG heart waveforms was then decomposed (step 105). Features of the PPG heart waveforms were then extracted using the first ML process (step 106), and further filtered (step 109). Over 3000 features were extracted, including the categories explained above, for each PPG waveform and stored. For purposes of the experiments, only the first ML process was implemented at this stage of the experiment.

Standing and supine position data was included in the development of the referential hydration and heat stress values and indices (steps 110, 111, and 112).

To implement the first ML process an XGBoost ML process was used (e.g., an algorithm). To generate hydration and heat stress values and combined hydration and heat stress index data was processed using linear regression and Bayesian statistical processes (e.g., algorithms; step 113).

Experimental, referential (1) hydration, (2) heat stress, and (3) combined hydration and heat stress indices were generated, with the following values being defined:

Heat=Increase in Core Temp for current Trial/Maximum Achieved Increase in Core Temp over all Trials

Hydr=Increase in WgtLoss for current Trial/Maximum Achieved Increase in WgtLoss over all Trials

HHI=(Heat+Hydr)/2

In addition, variables were defined as follows:
CoreTemp: observed core body temperature
Wgt (weight): measured nude body mass
WgtLost (weight loss): measured change in nude body mass

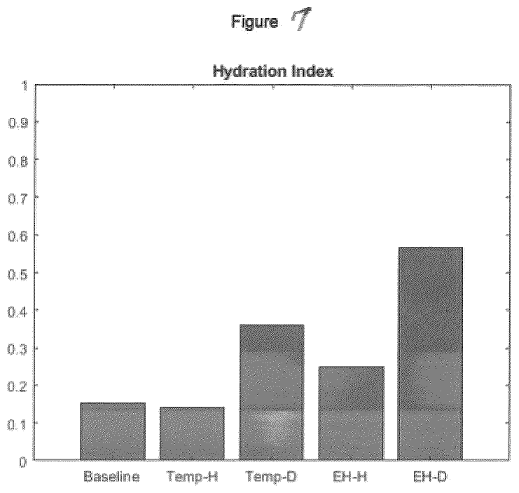

To test whether the referential values and indices developed using process/method 100 were valid, a leave-one-out cross-validation process was completed using process/method 200. The resulting mean hydration index values for all of the individuals involved in the experiments for each of the states mentioned before are shown in FIG. 7. From FIG. 7, the predicted hydration index values rose for each increased level of dehydration as an individual is subjected to a Temp-H state and then a Temp-D state, or as an individual is subjected to an EH-H state and then an EH-D state. It should also be noted that the experimental, mean hydration index was highest in a high-heat, dehydrated state (EH-D).

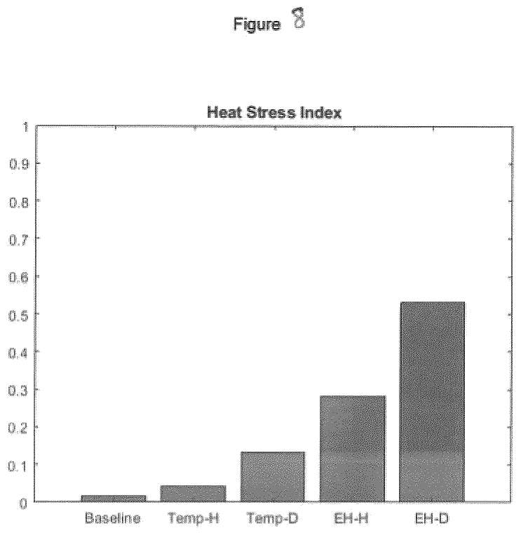

Similarly, mean heat stress indices and values for all of the individuals involved in the experiments were computed again utilizing the steps of method 200 (see FIG. 8).

The observed heat stress Indices (i.e., values), as shown in FIG. 8, were higher for individuals while being subjected to a Temp-D state as compared to when the individuals where subjected to the Temp-H state. The inventors believe that this indicates an increase in core body temperature after exercise in a dehydrated state. Further, it should be noted that the value of the heat stress index is higher while an individual is in a high-temperature state (e.g., EH-H and EH-D) as compared to a heat stress index while the individual is subjected to a room temperature state (e.g., Baseline, Temp-H, Temp-D). Still further, the value of a heat stress index is the highest when an individual is dehydrated in a high-temperature state (e.g., EH-D).

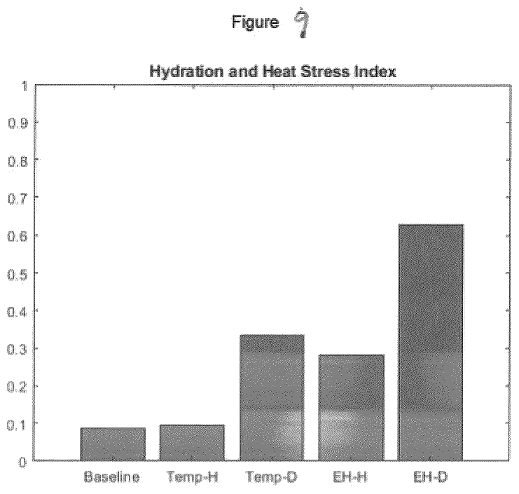

A combined hydration and heat stress Index was also evaluated, utilizing method 200 (see FIG. 9). As depicted in FIG. 9, the values of the hydration and heat stress index are: (a) higher for the dehydrated states (e.g., Temp-D and EH-D) as compared with the Baseline state; or (b) when a similar state is subjected to a higher temperature level compared to a lower temperature level (e.g., Temp-D versus and Temp-H, and EH-D versus and EH-H) (c) higher for high heat states (EH-H and EH-D) as compared with the Baseline state, and (d) the highest for the high heat and dehydrated state (EH-D).

The exemplary hydration Index, heat stress Index, and combined hydration and heat stress Indices provide a valuable relative measure of hydration and body temperature.

In additional embodiments the hydration, heat stress and combined hydration/heat stress levels and states generated by method 200 (and device 200a) may be used to provide specific, as compared to relative, values to a user. For example, one or more of the hydration and heat stress levels and states may be combined with personalized data to generate definitive, specific values that a user may use to adjust his or her hydration level or heat stress level (e.g., how many milliliters of water must I drink to reach a hydration level?).

Method 200 may include step 217, where the one or more processors 4a to 4n are operable to complete the processes that comprise the hydration and heat stress interpretive model described above with respect to step 113 in method 100.

In an embodiment, a plurality of signal data based values from one or more of steps 201 to 213 may be input into step 217. Each of the plurality of values may be correlated to (i) current hydration levels and states and patterns, and current heat stress states and patterns and (ii) current, personalized hydration and heat stress levels and states to substantially simultaneously determine one or more specific hydration and/or heat stress levels and states as well as information regarding how to achieve a specific hydration or heat stress level. In embodiments, step 217 may comprise identifying a specific hydration and.or heat stress level or amount (e.g., how many millimeters of water should an individual drink after exercising) that most closely correlate with each of the the plurality of inputted values.

The inventors believe that the ability to identify specific hydration and/or heat stress levels, rather than relative physiological states, is innovative.

The identified, specific hydration and/or heat stress may be stored in storage devices 5a to 5n in step 218.

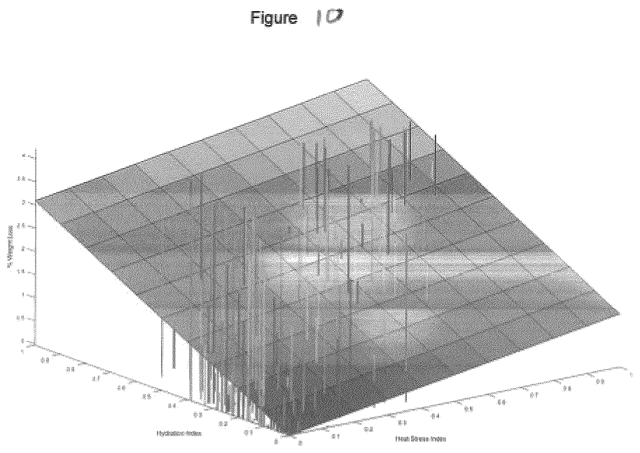

Referring to FIG. 10 there is depicted a three dimensional representation (e.g., a planar 3D contour) that may be used to predict a percentage of fluid weight loss for an individual given different hydration index and heat index values, among other things, based on completing an exemplary interpretative process following a Bayesian process (e.g., algorithm).

FIG. 10 also includes vertical bars that are based on the plurality of test individuals, described previously above, used in developing the three dimensional representation.

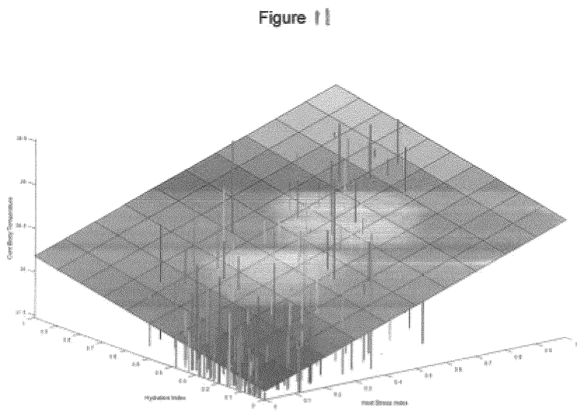

Similarly, FIG. 11 depicts a three dimensional representation (e.g., again a planar 3D contour) that may be used to predict a change in a core body temperature for an individual given different hydration index and heat index values, among other things, based on completing an exemplary interpretative process, also following a Bayesian process, and vertical bars that are based on the plurality of test individuals described previously above.

The exemplary interpretative process that utilizes both hydration and heat stress levels, as well as personalization data, used to generate the representations depicted in FIGS. 10 and 11 may be represented by the following relationships:

wtloss or temp=subjects*(α*heat+β*hydr+γ*heat*hydr) (1)

where δ are values from a biographical model capturing personalization data of an individual or individuals (e.g., sex, height, mass, and body fat, as well as hydration and heat tolerance measures) and α β γ are values that are common to all individuals.

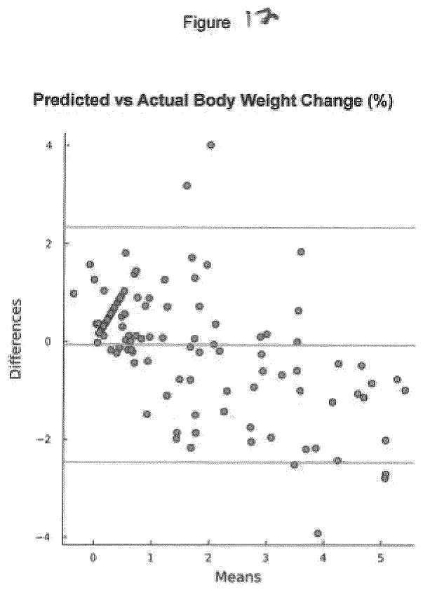

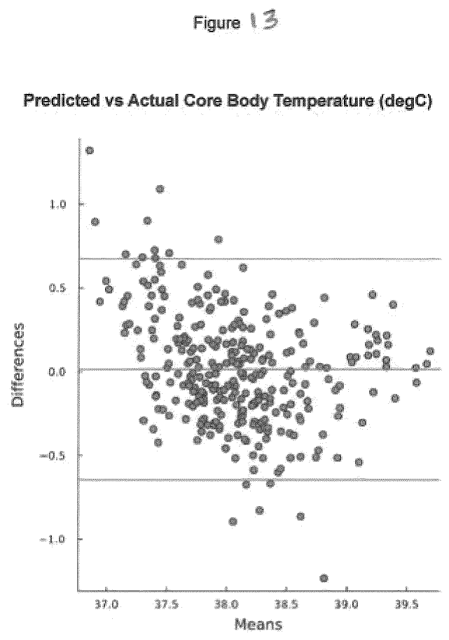

FIG. 12 depicts a Bland-Altman plot that compares predicted body weight loss percentages versus actual body weight loss percentages for different stages of the 26 subjects based on the interpretative process (model) discussed herein. Similarly, FIG. 13 shows a Bland-Altman plot that compares predicted changes in core body temperatures versus actual changes in core body temperatures based on the interpretative process.

From the foregoing description, one skilled in the art can easily ascertain the essential characteristics of the described embodiments. Further, it should be understood that the foregoing description only describes a few of the many possible embodiments that fall within the scope of the disclosure. Accordingly, those skilled in the art may make numerous changes and modifications to the embodiments disclosed herein without departing from the general spirit of the disclosure.

We claim:

1. A method for estimating physiological levels or states comprising:
electronically receiving a plurality of electronic signals representing photoplethysmography (PPG) and/or electrocardiogram (ECG) waveforms;
electronically explicitly or implicitly extracting one or more features of the PPG and/or electrocardiogram (ECG) waveforms from the plurality of electronic signals to generate values indicating one or more physiological characteristics of the PPG and/or electrocardiogram (ECG) waveforms; and
electronically correlating the generated values to one or more (i) physiological levels and sates, (ii) relative, hydration levels and states, or (iii) relative heat stress levels and states or (iv) a combination of relative, hydration and heat stress levels and states.

2. The method of claim 1 wherein electronically extracting the one or more features of the PPG waveforms and/or ECG waveforms from the plurality of electronic signals comprises extracting features from one or more categories of features of the PPG waveforms and/or ECG waveforms.

3. The method as in claim 2 wherein the one or more categories of features comprises at least one or more features selected from at least power features, wavelet features, and distribution features of the one or more PPG waveforms and/or ECG waveforms.

4. The method of claim 1 further comprises electronically selecting certain segments of the received signals representing certain points that are equidistant apart on each of the PPG and/or ECG waveforms, and further electronically, implicitly extracting one or more features of the PPG and/or ECG waveforms based on the selected, certain segments to generate the values indicating the one or more physiological characteristics of the PPG waveforms.

5. The method of claim 1 further comprising electronically excluding corrupted signals within the received electronic signals for an associated time segment during which the corrupted signals were received when one or more extracted features of the received electronic signals and/or an entirety of the received electronic signals is outside an acceptable range as compared to previously observed electronic signals of the same type within a past finite time period stored in an electronic database.

6. The method of claim 1 further comprising electronically adjusting the generated values by skewing, scaling or rotating one or more signals so that the generated values more closely reflect a representation of the received signals representing the PPG and/or ECG waveforms.

7. The method of claim 1 further comprising electronically generating one or more control signals to control the output of the one or more (i) relative, hydration levels and states, or (ii) relative heat stress levels and states or (iii) a combination of relative, hydration and heat stress levels and states or electronically generating one or more alarm indicators indicating one of the one or more (i) relative, hydration levels and states, or (ii) relative heat stress levels and states or (iii) a combination of relative, hydration and heat stress levels and states has exceeded a threshold.

8. The method of claim 1 further comprising electronically receiving personalization data; and further electronically correlating the generated values to one or more (i) absolute, hydration values, (ii) absolute, skin or core body temperature values, or (iii) a combination of absolute, hydration values and skin or core body temperature values.

9. An electronic device that estimates physiological levels or states comprising:
one or more electronic processors operable to execute instructions retrieved from one or more electronic memories to:
receive a plurality of electronic signals representing photoplethysmography (PPG) and/or electrocardiogram (ECG) waveforms;
electronically extract one or more features of the PPG and/or ECG waveforms from the plurality of electronic signals to generate values indicating one or more physiological characteristics of the PPG and/or ECG waveforms; and
electronically correlate the generated values to one or more (i) physiological levels and states, (ii) relative, hydration levels and states, or (iii) relative heat stress levels and states or (iv) a combination of relative, hydration and heat stress levels and states.

10. The electronic device as in claim 9 wherein the one or more electronic processors are further operable to execute instructions retrieved from the one or more electronic memories to electronically extract the one or more features of the PPG waveforms from the plurality of electronic signals comprises extracting features from one or more categories of features of the PPG and/or ECG waveforms.

11. The electronic device as in claim 9 wherein the one or more categories of features comprises at least one or more features selected from at least power features, wavelet features, and distribution features of the one or more PPG and/or ECG waveforms.

12. The electronic device as in claim 9 wherein the one or more electronic processors are further operable to execute instructions retrieved from the one or more electronic memories to electronically select certain segments of the received signals representing certain points that are equidistant apart on each of the PPG and/or ECG waveforms, and further electronically, implicitly extract one or more features of the PPG and/or ECG waveforms based on the selected, certain segments to generate the values indicating the one or more physiological characteristics of the PPG and/or ECG waveforms.

13. The electronic device as in claim 9 wherein the one or more electronic processors are further operable to execute instructions retrieved from the one or more electronic memories to electronically exclude corrupted signals within the received electronic signals for an associated time segment during which the corrupted signals were received when one or more extracted features of the received electronic signals and/or an entirety of the received electronic signals is outside an acceptable range as compared to previously observed electronic signals of the same type within a past finite time period stored in an electronic database.

14. The electronic device as in claim 9 wherein the one or more electronic processors are further operable to execute instructions retrieved from the one or more electronic memories to electronically adjust the generated values by skewing, scaling or rotating one or more signals so that the generated values more closely reflect a representation of the received signals representing the PPG and/or ECG waveforms.

15. The electronic device as in claim 9 wherein the one or more electronic processors are further operable to execute instructions retrieved from the one or more electronic memories to electronically generate one or more control signals to control the output of the one or more (i) relative, hydration levels and states, or (ii) relative heat stress levels and states or (iii) a combination of relative, hydration and heat stress levels and states or electronically generate one or more alarm indicators indicating one of the one or more (i) relative, hydration levels and states, or (ii) relative heat stress levels and states or (iii) a combination of relative, hydration and heat stress levels and states has exceeded a threshold.

16. The electronic device as in claim 9 wherein the one or more electronic processors are further operable to execute instructions retrieved from the one or more electronic memories to electronically:
receive personalization data; and
further electronically correlate the generated values to one or more (i) absolute, hydration values, or (ii) absolute, skin or core body temperature values, or (iii) a combination of absolute, hydration values and skin or core body temperature values.
